(19) United States
(12) Patent Application Publication
(10) Pub. No.: US 2018/0340937 A1
(43) Pub. Date: Nov. 29, 2018

(54) Phosphoinositide (4,5) Bisphosphate as a Diagnostic Tool and Target for Cancer Treatment

(71) Applicant: Christopher Janetopoulos, Nashville, TN (US)

(72) Inventor: Christopher Janetopoulos, Nashville, TN (US)

(21) Appl. No.: 15/963,157

(22) Filed: Apr. 26, 2018

Related U.S. Application Data
(63) Continuation of application No. 15/067,874, filed on Mar. 11, 2016, now abandoned.
(60) Provisional application No. 62/131,765, filed on Mar. 11, 2015.

(51) Int. Cl. G01N 33/574; G01N 33/92

(52) U.S. Cl. CPC G01N 33/57484; G01N 33/92

(57) ABSTRACT

The present invention relates to PI(4,5)P2 and more particularly to use of enzymes that metabolize and regulate the PI(4,5)P2 levels as a as a target for cancer treatment. Furthermore, measuring the level of PI(4,5)P2 or the degree of turnover of PI(4,5)P2 in cancer cells is an important diagnostic tool.

### PRIOR APPLICATIONS

This application claims priority to U.S. Ser. No. 62/131,765 filed Mar. 11, 2015, which is hereby incorporated in its entirety into this application.

### STATEMENT REGARDING FEDERALLY FUNDED RESEARCH

invention was made with government support under grant number R01GM080370 awarded by NIH. The government has certain rights in the invention.

### FIELD OF THE INVENTION

The present invention relates to phosphoinositide 4,5 bisphosphate (PI(4,5)P2) and more particularly to the use of agents that modulate PI(4,5)P2 as a target for cancer treatment by obstructing the metastatic potential of a cancer, and assaying levels of PI(4,5)P2.

### BACKGROUND

PI(4,5)P2 is a phospholipid present in the cell membrane, is a substrate for a number of enzymes in biochemical pathways, such as the important role in the IP3/DAG cellular signaling pathway. PI(4,5)P2 is hydrolyzed by the phosphoinositide phospholipase C (PI-PLC), a membrane-bound enzyme, when binding of cognate ligand to G protein-coupled receptors activates the Ga subunit of G protein coupled receptors. The hydrolysis of PI(4,5)P2 generates inositol 1,4,5-trisphosphate and diacylglycerol (DAG), both of which function as second messengers. DAG remains on the cell membrane and activates the signal cascade by activating protein kinase C (PKC). PKC in turn phosphorylates cytosolic proteins and modulates their activities. Inositol 1,4,5-trisphosphate induces mobilization of calcium Ca²⁺ channels into the cytosol by activating its receptors on the smooth endoplasmic reticulum (ER) which in turn open calcium channels. Calcium participates in the cascade by activating other proteins.

### SUMMARY

As described herein, it was surprisingly found that the precise regulation of the spatial and temporal levels of PI(4,5)P2 are critical for cell polarity during cell migration and division, both of which play a role in metastasis. Importantly, it was also found that Ras activity is inhibited by high PI(4,5)P2 levels, which sets up a powerful negative feedback loop that regulates another class of enzymes that use PI(4,5)P2 as a substrate, the P I 3′ Kinases (PI3Ks). The activation of PI3Ks leads to elevation of PI(3,4,5)P3, while simultaneously lowering the substrate, PI(4,5)P2, and this upregulation of PI(3,4,5)P3 is also associated with highly metastatic cancers. Carmeno, The PKB/AKT pathway in cancer, Current pharmaceutical design 16(1):34-44, 2010; Yuan and Cantley, PI3K pathway alterations in cancer: variations on a theme, Oncogene 27(41):5497-510; 2008; Cantley, The phosphoinositide 3-kinase pathway, Science 296(5573):1655-1657, 2002. It was also determined that low PI(4,5)P2 levels contribute to increased actin polymerization, resulting in more leading edge projections. This increases invasiveness, the migration of cells, which would contribute to metastasis of cancer cells. Cells that have an active actin cytoskeleton and are sending out protrusive structures, may also have problems during cell division, which can result in aneuploidy and further contribute to the mutation rate of cancers. Importantly, as disclosed herein, it was surprisingly determined that stimulation of a cell with a chemoattractant initially lowered the PI(4,5)P2 levels, but other feedback loops rapidly readjusted the PI(4,5)P2 levels to their prestimulus level. Thus, the results described herein indicate that mutations that lead to local low PI(4,5)P2 levels, or that rapidly turnover PI(4,5)P2, as compared to levels in normal cells, results in more invasive and metastatic cancer cells. Thus, in one aspect, cancer cells having lower PI(4,5)P2 levels than normal, and/or rapidly turning over PI(4,5)P2 are likely highly metastatic. In some aspects, a determination that a subject with cancer has low PI(4,5)P2 levels or rapid turnover of PI(4,5)P2 levels may result in a clinician prescribing more aggressive treatments for the subject. In one aspect, this disclosure relates to methods and tools for measuring PI(4,5)P2 levels and rapid turnover, useful in diagnosis and classification of cancer.

In some aspects, this disclosure relates to a method of treating cancer by administering an effective amount of an up modulating agent that raises PI(4,5)P2 levels, for example, restores the PI(4,5)P2 levels to approximately normal levels. In some embodiments, the administering of an effective amount of an up-modulating agent of PI(4,5)P2 decreases the invasiveness, migration and/or metastasis of cancer cells and can be used to treat the cancer, or to prevent or treat metastasis. In some aspects, cancers with lower levels of PI(4,5)P2 than normal cells may be treated with agents that increase the levels of PI(4,5)P2 (up modulating agents). Any upregulating agent as disclosed herein may be used in these methods.

It was also recognized that although mutations that would be predicted to lower PI(4,5)P2 level, such as mutation or deletions in the tumor suppressor PTEN, or mutations that activate the small GTPases such as Ras are common in cancers, but mutations that both lower PI(4,5)P2 levels are uncommon. In the later case, activating Ras would activate PI3Ks, which in turn would deplete the local cellular levels of the substrate PI(4,5)P2. This sets up a positive feedback loop that further activates the Ras. In an alternative aspect, this invention provides a method of treating cancer by administering to cancer cells having lower levels of PI(4,5)P2 compared to normal cells, an agent that further lowers PI(4,5)P2 level in the cancer cells compared to normal cells. In one aspect, administering the PI(4,5)P2 down modulating agent that further lowers PI(4,5)P2 levels prevents further cell division, and can be used to treat cancer, or to prevent or treat metastasis. In some aspects, this invention provides a method of treating cancer by administering an effective amount of a down modulating agent, that is, an agent that further lowers PI(4,5)P2 levels and selectively kills the cancer cells.

Thus, in some aspects, this disclosure provides a method of treating cancer in a subject comprising administering a therapeutically effective amount of an agent that modulates the level of PI(4,5)P2 in a cancer cell of the subject. In some aspects, this invention provides a method of treating cancer by administering a therapeutically effective amount of a modulating agent that lowers the PI(4,5)P2 level in a subject in need thereof. Any downregulating agent as disclosed herein may be used in these methods.

In some aspects, this disclosure relates to modulating the levels of PI(4,5)P2 by modulating one or more enzymes that produce PI(4,5)P2 or one or more enzymes that use PI(4,5)P2 as a substrate. In some embodiments, the agent is a modulator of an enzyme selected from the group consisting of a phosphatidylinositol 4 phosphate 5 kinase, a phosphatidylinositol 5 phosphate 4 kinase, a phosphatidylinositol-3,4,5-trisphosphate 3-phosphatase (the tumor suppressor PTEN), a phosphatidylinositol-4,5-bisphosphate 3-kinase, a SH2-containing inositol phosphatase (SHIP) and a phosphatidylinositol phospholipase C (collectively, “the enzymes modulating PI(4,5)P2” hereinafter). In some aspects the PI(4,5)P2 modulating agent is selected from the group consisting of a stimulator or agonist of the enzymes, or an antagonist, blocker or inhibitor of the enzymes. In some aspects the PI(4,5)P2 modulating agent may be an agent that increases or decreases the transcription, expression, or activity of one or more enzymes that produce PI(4,5)P2 or one or more enzymes that use PI(4,5)P2 as a substrate.

In some aspects, this disclosure relates to modulating the levels of PI(4,5)P2 by modulating one or more enzymes that produce PI(4,5)P2. In some aspects, this disclosure relates to modulating the levels of PI(4,5)P2 by modulating one or more enzymes that produce PI(4,5)P2 selected from the group consisting of a phosphatidylinositol 4 phosphate 5 kinase, a phosphatidylinositol 5 phosphate 4 kinase, and a phosphatidylinositol-3,4,5-trisphosphate 3-phosphatase (the tumor suppressor PTEN) (collectively, “the enzymes that produce PI(4,5)P2”).

In some aspects, this disclosure relates to up-modulating the levels of PI(4,5)P2 by administering to a subject in need thereof an effective amount of a modulator that increases the activity level or amount of one or more enzymes that produce PI(4,5)P2, thereby upregulating PI(4,5)P2. In some aspects an up modulator of an enzyme that produces PI(4,5)P2 is selected from the group consisting of stimulator or agonist. In some aspects the PI(4,5)P2 up-modulating agent may be an agent that increases the transcription, expression, or activity of one or more enzymes that produce PI(4,5)P2.

In other aspects, this disclosure relates to down-modulating the levels of PI(4,5)P2 by administering to a subject in need thereof an effective amount of a modulator that decreases the activity level or amount of one or more enzymes that produce PI(4,5)P2, thereby downregulating PI(4,5)P2. In some aspects a down-modulator of an enzyme that produces PI(4,5)P2 is selected from the group consisting of an antagonist, blocker, inhibitor or other downregulator of one or more enzymes that produce PI(4,5)P2. In some aspects the PI(4,5)P2 down-modulating agent may be an agent that decreases the transcription, expression, or activity of one or more enzymes that produce PI(4,5)P2.

In some aspects, this disclosure relates to modulating the levels of PI(4,5)P2 by modulating one or more enzymes that use PI(4,5)P2 as a substrate. In some aspects, this disclosure relates to modulating the levels of PI(4,5)P2 by modulating one or more enzymes that use PI(4,5)P2 as a substrate, wherein the enzyme is selected from the group consisting of a phosphatidylinositol-4,5-bisphosphate 3-kinase, a SH2-containing inositol phosphatase (SHIP) and a phosphatidylinositol phospholipase C (collectively, “the enzymes that metabolize PI(4,5)P2”). In some aspects, this disclosure relates to modulating the levels of PI(4,5)P2 by administering to a subject in need thereof a therapeutically effective amount of a modulator of one or more enzymes that metabolize PI(4,5)P2. In some aspects the modulator of the enzymes that metabolize PI(4,5)P2 is selected from the group consisting of stimulator, agonist, antagonist and inhibitor. In some aspects the modulator of the enzymes that metabolize PI(4,5)P2 may be an agent that increases or decreases the transcription, expression, or activity of one or more enzymes that metabolize PI(4,5)P2.

In some aspects, this disclosure relates to down modulating the levels of PI(4,5)P2 by up regulating or up modulating, e.g., stimulating or activating one or more enzymes that metabolize PI(4,5)P. In other aspects, this disclosure relates to up modulating the levels of PI(4,5)P2 by down regulating, e.g., blocking, inhibiting and/or antagonizing, one or more enzymes that metabolize PI(4,5)P2. In some aspects the PI(4,5)P2 down-modulating agent may be an agent that increases the transcription, expression, or activity of one or more enzymes that metabolize PI(4,5)P2.

In some aspects, the method further comprises screening a cancer patient to identify a suitable treatment, the method comprising (i) providing a sample from the subject and a control sample; and (ii) testing the sample to detect one or more of (a) level of phosphatidylinositol-4,5-bisphosphate, (b) mutation in a gene encoding an enzyme modulating PI(4,5)P2; and (c) activity of an enzyme selected from the group consisting of the enzymes modulating PI(4,5)P2. In some embodiments the sample comprises, for example, plasma membranes or fragments thereof, and may be any sample as described herein.

In some aspects, when the level of PI(4,5)P2 in a sample from a subject is low or if the PI(4,5)P2 is rapidly turning over, the agent used for treating cancer in the subject is an agent that modulates the enzymes that produce or metabolize PI(4,5)P2. In some embodiments, when the level of PI(4,5)P2 in a sample from a subject is low, the agent used for treating cancer in the subject is a modulator of the enzymes that produce PI(4,5)P2. In some embodiments, when the level of PI(4,5)P2 in a sample from a subject is low, the agent used for treating cancer in the subject is a modulator of the enzymes that metabolize PI(4,5)P2.

In some aspects, when the level of PI(4,5)P2 in a sample from a subject is low or where the PI(4,5)P2 is rapidly turning over, the agent used for treating cancer in the subject is an agent that increases the level of PI(4,5)P2. In some embodiments, the agent used for treating cancer in the subject is a stimulator or an agonist of enzymes that produce PI(4,5)P2, or an inhibitor or an antagonist of enzymes that metabolize PI(4,5)P2.

In some alternative aspects, when the level of PI(4,5)P2 in a sample from a subject is low or where the PI(4,5)P2 is rapidly turning over, the agent used for treating cancer in the subject is an agent that further decreases the level of PI(4,5)P2. In some embodiments, the agent used for treating cancer in the subject is an inhibitor or an antagonist of enzymes that produce PI(4,5)P2, or a stimulator or an agonist of enzymes that metabolize PI(4,5)P2.

In some embodiments, when a subject has a mutation in a gene encoding a phosphatidylinositol 4 phosphate 5 kinase or reduced activity of a phosphatidylinositol 4 phosphate 5 kinase, the agent used for treating cancer in the subject is a down modulator, inhibitor, blocker or antagonist of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some embodiments, when a subject has a mutation in a gene encoding a phosphatidylinositol 4 phosphate 5 kinase or reduced activity of a phosphatidylinositol 4 phosphate 5 kinase, the agent used for treating cancer in the subject is an upregulator, agonist or stimulator of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some embodiments, when a subject has a mutation in a gene encoding a phosphatidylinositol 4 phosphate 5 kinase or reduced activity of a phosphatidylinositol 4 phosphate 5 kinase, the agent used for treating cancer in the subject is a down modulator, inhibitor, blocker or antagonist of one or more of the enzymes that metabolize PI(4,5)P2.

In some alternative embodiments, when a subject has a mutation in a gene encoding a phosphatidylinositol 4 phosphate 5 kinase or reduced activity of a phosphatidylinositol 4 phosphate 5 kinase, the agent used for treating cancer in the subject is an upregulator, agonist or stimulator of one or more of the enzymes that metabolize PI(4,5)P2.

In some embodiments, when a subject has a mutation in a gene encoding a phosphatidylinositol 5 phosphate 4 kinase or reduced activity of a phosphatidylinositol 5 phosphate 4 kinase, the agent used for treating cancer in the subject is a down modulator, inhibitor, blocker or antagonist of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some alternative embodiments, when a subject has a mutation in a gene encoding a phosphatidylinositol 5 phosphate 4 kinase or reduced activity of a phosphatidylinositol 5 phosphate 4 kinase, the agent used for treating cancer in the subject is an upregulator, agonist or stimulator of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some embodiments, when a subject has a mutation in a gene encoding a phosphatidylinositol 5 phosphate 4 kinase or reduced activity of a phosphatidylinositol 5 phosphate 4 kinase, the agent used for treating cancer in the subject is a down modulator, inhibitor, blocker or antagonist of one or more of the enzymes that use PI(4,5)P2 as a substrate.

In some embodiments, when a subject has a mutation in a gene encoding a phosphatidylinositol 5 phosphate 4 kinase or reduced activity of a phosphatidylinositol 5 phosphate 4 kinase, the agent used for treating cancer in the subject is an upregulator, agonist or stimulator of one or more of the enzymes that use PI(4,5)P2 as a substrate.

In some embodiments, when a subject has a mutation in a gene encoding a phosphatidylinositol-3,4,5-trisphosphate 3-phosphatase or reduced activity of a phosphatidylinositol-3,4,5-trisphosphate 3-phosphatase, is a down modulator, inhibitor, blocker or antagonist of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some alternative embodiments, when a subject has a mutation in a gene encoding a phosphatidylinositol-3,4,5-trisphosphate 3-phosphatase or reduced activity of a phosphatidylinositol-3,4,5-trisphosphate 3-phosphatase, is an upregulator, agonist or stimulator of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some embodiments, when a subject has a mutation in a gene encoding a phosphatidylinositol-3,4,5-trisphosphate 3-phosphatase or reduced activity of a phosphatidylinositol-3,4,5-trisphosphate 3-phosphatase, the agent used for treating cancer in the subject is a down modulator, inhibitor, blocker or antagonist of one or more of the enzymes that use PI(4,5)P2 as a substrate.

In some alternative embodiments, when a subject has a mutation in a gene encoding a phosphatidylinositol-3,4,5-trisphosphate 3-phosphatase or reduced activity of a phosphatidylinositol-3,4,5-trisphosphate 3-phosphatase, the agent used for treating cancer in the subject is a upregulator, agonist or stimulator of one or more of the enzymes that use PI(4,5)P2 as a substrate.

In some embodiments, assaying a sample from the subject is used to detect that the subject has a mutation or epigenetic alterations in one or more the genes encoding enzymes that produce or use PI(4,5)P2. Any method described herein to detect a mutation or in the gene may be used to assay the sample. Methods to determine the levels, expression or post translational modification of enzymes that produce or use PI(4,5)P2 may also be used.

In some embodiments the method used to determine whether the subject has a mutation in an enzyme that produces PI(4,5)P2 or uses PI(4,5)P2 as a substrate comprises the use of one or more of the following methods: quantitative PCR (q-PCR), reverse transcription polymerase chain reaction (RT-PCR), real-time PCR (RT-PCR), polyacrylamide gel electrophoresis (PAGE), multiplex ligation dependent probe amplification (MLPA), sanger sequencing, targeted DNA sequencing, whole genome DNA sequencing, exome sequencing, pyrosequencing, comparative genomic hybridization array (CGH), restriction fragment length polymorphism (RFLP), short tandem repeat analysis (STR), dynamic allele-specific hybridization (DASH), genotyping, variable number tandem repeats (VNTRs), molecular beacons, amplification refractory mutation system PCR (ARMS-PCR), Invader™ assay (flap endonuclease (FEN)), primer extension, mass spectrometry, TaqMan™, denaturing high performance liquid chromatography (DHPLC), and high resolution melting analysis.

In some aspects of this disclosure, the control sample is a normal tissue sample from the subject, a biological sample derived from a normal subject or a solution comprising phosphatidylinositol-4,5-bisphosphate. In some embodiments the tissue sample may be a plasma membrane sample, or a sample containing, obtained from or derived from plasma membranes or fragments thereof. In some embodiments the control sample may be a tissue sample from a non-cancerous tissue of the subject. In some embodiments, the cancer or control tissue sample from the subject is selected from the group consisting of blood, serum, plasma, lymph, urine, saliva, a mucosal secretion, a vaginal secretion, cerebrospinal fluid, serosal fluid, ascites fluid, pleural fluid, pericardial fluid, peritoneal fluid, abdominal fluid, lavage fluid, fecal matter, sputum, biopsy sample, autopsy sample, tears, washings obtained during a medical procedure. The sample may also be a processed sample, for example, extracted, isolated or purified lipids, DNA, protein, nucleic acids, amino acids, metabolites, analytes, and/or conditioned culture medium used for cultivating at least one cell from a subject and a cell lysate.

In some embodiments, the modulator of the enzymes modulating PI(4,5)P2, used for treating cancer, is selected from the group consisting of a chemical inhibitor, an antagonist, a chemical modulator, a chemical stimulator, an agonist, a polynucleotide-based agent affecting transcription or translation of the phosphatidylinositol 4 phosphate 5 kinase, and a mixture thereof. In some embodiments, the modulator of the enzymes that produce PI(4,5)P2, used for treating cancer, is selected from the group consisting of a chemical inhibitor, an antagonist, a chemical modulator, a chemical stimulator, an agonist, a polynucleotide-based agent affecting transcription or translation of the phosphatidylinositol 4 phosphate 5 kinase and a mixture thereof. In some embodiments, the modulator of the enzymes that use PI(4,5)P2, used for treating cancer, is selected from the group consisting of a chemical inhibitor, an antagonist, a chemical modulator, a chemical stimulator, an agonist, a polynucleotide-based agent affecting transcription or translation of the phosphatidylinositol 4 phosphate 5 kinase and a mixture thereof.

In some embodiments, the modulator of a phosphatidylinositol 4 phosphate 5 kinase, used for treating cancer, is selected from the group consisting of a chemical inhibitor, an antagonist, a chemical modulator, a chemical stimulator, an agonist, a polynucleotide-based agent affecting transcription or translation of the phosphatidylinositol 4 phosphate 5 kinase and a mixture thereof. In some embodiments, the modulator of a phosphatidylinositol 5 phosphate 4 kinase, used for treating cancer, is selected from the group consisting of a chemical inhibitor, an antagonist, a chemical modulator, a chemical stimulator, an agonist, a polynucleotide-based agent affecting transcription or translation of the phosphatidylinositol 5 phosphate 4 kinase and a mixture thereof. In some embodiments, the modulator of a phosphatidylinositol-4,5-bisphosphate 3-kinase, used for treating cancer, is selected from the group consisting of a chemical inhibitor, an antagonist, a chemical modulator, a chemical stimulator, an agonist, AEZS-136, BAY 80-6946, BEZ235, BKM120, CAL263, CUDC-907, demethoxyviridin, GNE-477, GSK1059615, IC87114, idelalisib, INK1117, IPI-145, LY29400, Palomid 529, perifosine, PI-103, PWT33597, PX-866, RP6503, RP6530, SF1126, TG100-115, TGR 1202, wortmannin, XL147 (SAR245408), XL765 (SAR245409), ZSTK474 a polynucleotide-based agent affecting transcription or translation of the phosphatidylinositol-4,5-bisphosphate 3-kinase C and a mixture thereof. In some embodiments, the modulator of a phosphoinositide phospholipase C, used for treating cancer, is selected from the group consisting of a chemical inhibitor, an antagonist, a chemical modulator, a chemical stimulator, an agonist, D609(Tricyclodecan-9-yl-xanthogenate), edelfosine, U73122, a polynucleotide-based agent affecting transcription or translation of the phosphoinositide phospholipase C and a mixture thereof. In some embodiments, the modulator of a phosphatidylinositol-3,4,5-trisphosphate 3-phosphatase, used for treating cancer, is selected from the group consisting of a chemical inhibitor, an antagonist, a chemical modulator, a chemical stimulator, an agonist, oncomiR, MIRN21, SF1670, a polynucleotide-based agent affecting transcription or translation of the phosphatidylinositol-3,4,5-trisphosphate 3-phosphatase (PTEN) and a mixture thereof. In some embodiments, the modulator of a SH2-containing inositol phosphatase (SHIP), used for treating cancer, is selected from the group consisting of a chemical inhibitor, an antagonist, a chemical modulator, a chemical stimulator, an agonist, a polynucleotide-based agent affecting transcription or translation of the SH2-containing inositol phosphatase (SHIP) and a mixture thereof.

Some aspects of this invention provide method of diagnosing cancer in a subject comprising:
(a) contacting a biological sample with a binding agent that that specifically binds to phosphatidylinositol-4,5-bisphosphate for sufficient time to form a first specific binding complex;
(b) removing constituents of the sample;
(c) contacting the first complex with a second binding agent to form a second specific binding complex;
(d) determining the level of phosphatidylinositol-4,5-bisphosphate in the sample by detecting the second complex;
wherein lower level of phosphatidylinositol-4,5-bisphosphate in the sample, compared to a control is correlated with cancer.

In some embodiments, the biological sample from a subject used for diagnosis of cancer is a sample comprising plasma membranes or portions thereof. In some embodiments the sample may be selected from the group consisting of blood, serum, plasma, lymph, urine, saliva, a mucosal secretion, a vaginal secretion, cerebrospinal fluid, serosal fluid, ascites fluid, pleural fluid, pericardial fluid, peritoneal fluid, abdominal fluid, lavage fluid, fecal matter, sputum, biopsy sample, autopsy sample, tears, washings obtained during a medical procedure and at least one cell derived from the subject. In some embodiments, the biological sample from a subject used for diagnosis of cancer is selected from the group consisting of at least one cell, culture medium, conditioned culture medium, cell lysate derived by culturing at least one cell derived from the subject.

In some embodiments, the first binding agent used for diagnosing cancer is selected from the group consisting of a polyclonal antibody, a monoclonal antibody, a bispecific antibody, a chimeric antibody, a humanized antibody, a single chain antibody, aptamer and a binding fragment of the polyclonal, monoclonal, bispecific, chimeric, humanized, or single chain antibody. In some embodiments, the second binding agent used for diagnosing cancer is selected from the group consisting of a polyclonal antibody, a monoclonal antibody, a bispecific antibody, a chimeric antibody, a humanized antibody, a single chain antibody, aptamer and a binding fragment of the polyclonal, monoclonal, bispecific, chimeric, humanized, or single chain antibody. In some embodiments, the first binding agent or the second binding agent, used for diagnosing cancer, comprises a detectable agent. In some embodiments, the first binding agent or the second binding agent, used for diagnosing cancer, comprises a detectable agent, wherein the detectable agent is selected from the group consisting of an enzyme, a fluorescent label, a chemiluminescent label, a nanoparticle label, a radioactive isotope, biotin, avidin and streptavidin.

In some embodiments, the method used for diagnosing cancer is an immunological method selected from the group consisting of ELISA, FACS, chemiluminescence, immunocytochemistry, radioimmunoassay, immunofluorescence microscopy, and western blotting. In some embodiments, the method used for diagnosing cancer, further comprises performing at least one additional diagnostic assay. In some embodiments, the method used for diagnosing cancer further comprises detecting increased Arp 2/3 actin polymerization.

In some aspects, the method of diagnosis of cancer in a subject comprises determination of the rate of turnover of PI(4,5)P2 levels in cancer cells.

In some embodiments, the method of diagnosis of cancer in a subject comprises:
(a) contacting a sample comprising at least one cancer cell with PI(4,5)P2 at an initial concentration, wherein the PI(4,5)P2 is labelled with a detectable marker;
(b) incubating the sample under a growth condition for an amount of time sufficient to cause turnover of the PI(4,5)P2;
(c) performing an assay to detect the level of one or more of the PI(4,5)P2, phosphatidylinositol-3,4,5-trisphosphate and inositol 1,4,5-trisphosphate;
(d) comparing the level of PI(4,5)P2, phosphatidylinositol-3,4,5-trisphosphate and inositol 1,4,5-trisphosphate, carrying the label, with the initial concentration;
(e) determining the rate of turnover of the PI(4,5)P2.

In some embodiments, the detectable marker used for labelling the phosphatidylinositol-4,5-bisphosphate includes but is not limited to radioactivity and a co-valently attached chemical reporter, wherein the reporter includes but is not limited to a fluorescent probe, a chemiluminiscent probe and a biotin.

In some embodiments, the assay used to detect one or more of the phosphatidylinositol-4,5-bisphosphate, phosphatidylinositol-3,4,5-trisphosphate and inositol 1,4,5-trisphosphate includes but is not limited to an immunological assay, a chromatography-based assay, a mass spectroscopy-based assay, an enzymatic assay.

### BRIEF DESCRIPTION OF THE DRAWINGS

FIG. 1 is the experimental set up for study of polarity reversals. To gain further insight into the mechanism regulating polarized morphology, polarity reversals were studied using the shown apparatus. Dictyostelium cells were lured into the PDMS (polydimethylsiloxane) channels by a 1 mM cAMP gradient. After the cells had entered the channel, the gradient was reversed by bringing a second micropipette to the rear of the cells. Cells completely broke down their polarity and reestablished it in the opposite direction.

FIG. 2 shows the response of cells to a uniform chemical stimulus. FIG. 2 represents fluorescent microscopy images of designated GFP tagged molecules before and after uniform addition of 100 μM folic acid, represented as “+Chemoattractant” on top of the images. Changes in plasma membrane localization of signaling and cytoskeletal elements in response to a stimulus can be used to predict their polarized localizations in a variety of processes. Molecules that fall off the plasma membrane are “back” proteins (left), while those that are recruited are typically “front” proteins (right).

FIG. 3 shows polarity reversal of cell expressing PTEN-GFP. Dictyostelium cells expressing the phosphatase and tumor suppressor PTEN were lured into the PDMS channels by a 1 mM cAMP gradient. After the cells had entered the channel, the gradient was reversed by bringing a second micropipette to the rear of the cells. Cells did not make a new front until PTEN levels dropped significantly. Extent of localization of PTEN mirrors the relative levels of phosphatidylinositol-4,5-bisphosphate.

FIG. 4 shows that PTEN localization and Ras activity are reciprocally regulated. Dictyostelium cells migrating to the bottom left corner that were stimulated with a uniform stimulus of 1 mM cAMP gradient (left). The Ras Bindng Domain of the Raf1, a marker for Ras activity, was recruited only to the front of the cell. Similarly, it was shown using 3D Lattice Sheet Illumination that PTEN localization and Ras activity are reciprocally regulated during random motility (right). These data indicates very powerful and negative feedback loops since high levels of phosphatidylinositol-4,5-bisphosphate appear to block Ras activity. This data indicates that mutations that lead to lower phosphatidylinositol-4,5-bisphosphate levels result in more invasive and metastatic cancer cells.

FIG. 5 shows the Local Excitation, Global Inhibition Model (LEGI) that explains cell responses to uniform stimulus and to a chemical gradient. Receptor occupancy during chemotaxis regulates two opposing processes, excitation and inhibition, which together regulate the response (green, red and black lines, respectively). When a cell is initially exposed to a gradient, both ends respond. The fast local excitation processes increase proportionally to the local fraction of occupied receptors. The slow inhibitory response rises, driven by the global fraction of occupied receptors. When both processes reach a steady state (Lower), the profile of excitation along the length of the cell is proportional to the local fraction, whereas the global inhibitor is proportional to the mean level of receptor occupancy, respectively. Thus, at the front, excitation exceeds inhibition, leading to a persistent response and vice versa at the rear.

FIG. 6 shows changes in phosphatidylinositol-4,5-bisphosphate in response to a uniform stimulus. Plasma membrane levels of phosphatidylinositol-4,5-bisphosphate drop rapidly in response to a uniform stimulus of cAMP as a number of enzymes that use phosphatidylinositol-4,5-bisphosphate as a substrate are activated. These low levels of phosphatidylinositol-4,5-bisphosphate contribute to the activation of ARP2/3 mediated actin responses. With a slight delay, phosphatidylinositol-4 and phosphatidylinositol 5 kinases are activated and begin to elevate the phosphatidylinositol-4,5-bisphosphate levels. PTEN, which contains a phosphatidylinositol-4,5-bisphosphate binding site is then recruited, which helps lower phosphatidylinositol-3,4,5-triphosphate levels and in a positive feedback loop, further raises phosphatidylinositol-4,5-bisphosphate. These high levels of phosphatidylinositol-4,5-bisphosphate are associated with formin-mediated actomyosin contraction. The signal transduction network adapts during continued stimulation (in part by phosphorylation events of receptors and other components) but a large part of the “inhibition” in the LEGI model is controlled by the enzymes that raise phosphatidylinositol-4,5-bisphosphate to amounts at or above prestimulus levels.

FIG. 7 shows changes in phosphatidylinositol phosphates are similarly regulated during cytokinesis. Localization of phosphatidylinositol-3-kinase and PTEN during anaphase in Dictyostelium (Far left, top and bottom) and cells before (−3) and after (3 sec) stimulation with chemoattractant are shown. Cells expressing a pleckstrin homology domain specific for phosphatidylinositol 4, 5-bisphosphate (PH-GFP) during the cell cycle. Phosphatidylinositol 4, 5-bisphosphate levels are localized in a reciprocal manner, as shown in Janetopoulos et al. Dev Cell 2005. This work supports the idea that ARP2/3-mediated actin polymerization takes place at the poles, and formin-mediated assembly in the furrow, where there is actomyosin contraction.

FIG. 8. Cells expressing iRAP sytem show PI(4,5)P2 depletion. Top, left: MDA-MB-231 cells before (−Rap) and approximately 1 minute after addition of 2 mM rapamycin (+Rap). RFP-Inp54p is recruited to the PM (arrows in +Rap). Left, middle: depletion of PM PI(4,5)P2 as PLC(d)-GFP redistributed to the cytosol (arrow showing enhanced cytosolic localization of PLC(d)). Bottom, left) Merged image of the two reporters. Top, right: Co-transfection of MDA-MB-231 cells with iRap system and GFP-F-tractin led to disappearance of stress fibers (arrow left), and its replacement with actin-filled projections (arrow right)˜2 mins after addition of rapamycin (See arrows). Middle, right: HUVECs transfection with iRap and GFP-F-tractin showing before and after rapamycin Note the formation of membrane protrusions (arrow, right). Bottom, right: Co-transfection of MDA-MB-231 cells with iRap system MyosinII-RLC and GFP-AldoA led redistribution of GFP-AldoA to the cytosol (Bottom right arrow show enhanced cytosolic localization and arrowhead show actin-filled protrusions)˜2 mins after addition of rapamycin (See arrows).

### DETAILED DESCRIPTION

Inositol is a carbohydrate naturally found in plants and animals. It exists in different phosphorylated and lipid-conjugated forms.

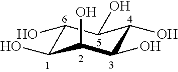

### Inositol

Inositol conjugated to diacylglycerol through a phosphate at position 1 are phosphatidylinositol. The lipids based on phosphatidylinositol are known as inositides, or sometimes phosphoinositides, which are usually phosphorylated at one or more positions Phosphatidylinositol 4,5-bisphosphate (also known as PI(4,5)P2, PtdIns(4,5)P2 or PIP2) is a phospholipid that is enriched at the plasma membrane The fatty acid chains present in PI(4,5)P2 differ between species and tissues, but stearic acid and arachidonic acid are very common at positions 1 and 2 of the glycerol moiety, respectively.

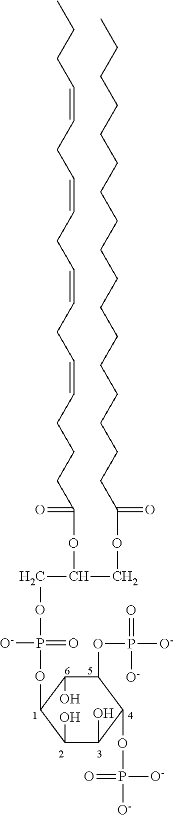

### Phosphatidylinositol 4,5-bisphosphate (PI(4,5)P2)

PI(4,5)P2 is formed by phosphorylation of either phosphatidylinositol 4-phosphate or phosphatidylinositol 5-phosphate, in the reactions catalyzed by phosphatidylinositol 4 phosphate 5 kinase and phosphatidylinositol 5 phosphate 4 kinase, respectively. It is also formed by dephosphorylation of phosphatidylinositol 3,4,5 trisphosphate, in a reaction catalyzed by the enzyme phosphatidylinositol-3,4,5-trisphosphate 3-phosphatase, also known as the tumor suppressor PTEN. PI(4,5)P2 is dephosphorylated to phosphatidylinositol 4-phosphate by the enzyme SHIP (SH2-containing inositol phosphatase), or phosphorylated to phosphatidylinositol 3,4,5 trisphosphate by the enzyme phosphatidylinositol-4,5-bisphosphate 3-kinases or cleaved by phosphoinositide phospholipase C.

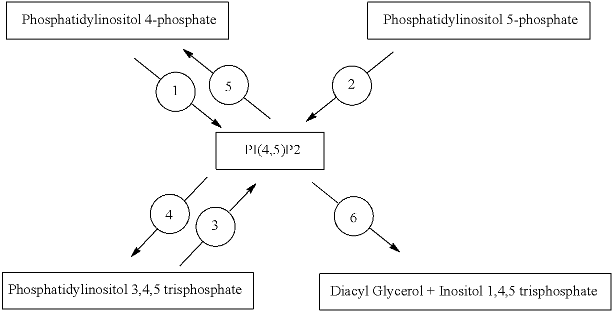

Enzymes involved in the metabolism of PI(4,5)P2. PI(4,5)P2 is produced by phosphatidylinositol 4 phosphate 5 kinase (shown as \textcircled{1}), phosphatidylinositol 5 phosphate 4 kinase (shown as \textcircled{2}) (and phosphatidylinositol-3,4,5-trisphosphate 3-phosphatase (the tumor suppressor PTEN, shown as \textcircled{3}. PI(4,5)P2 is used as a substrate, and thereby metabolized by phosphatidylinositol-4,5-bisphosphate 3-kinases (shown as \textcircled{4}), SH2-containing inositol phosphatase (SHIP) (shown as (\textcircled{5}) and phosphatidylinositol phospholipase C (shown as (\textcircled{6}).

As used herein “modulator” or “modulating agent” is a substance which directly or indirectly influences (modulates) the production, degradation, levels, and/or activity of an enzyme. A modulator may be an activator, agonist, antagonist or a stimulator of the enzyme, that alters Kᵢₙ of the enzyme for its substrate, Vₘₐₓ of the enzyme-catalyzed reaction, K_{cat} or other parameters that determine activity of the enzyme. These agents may bind the enzyme at the active site or allosterically, either reversibly or irreversibly, for example, by covalently binding to the enzyme. In some embodiments, a modulator may be an agent that alters the extent of transcription of the gene that encodes the enzyme, or alters the extent of translation of the mRNA transcribed from the gene that encodes the enzyme. In some embodiments, a modulator may be an agent that alters stability of the enzyme by either facilitating degradation or stabilizing the enzyme. The modulator may be a down-modulator if it decreases the activity of the enzyme by any mechanism described herein, for example by acting as an enzyme inhibitor, enzyme antagonist, inducer of enzyme degradation, inhibitor of transcription of gene encoding the enzyme, inhibitor of translation of the mRNA of the gene encoding the enzyme or a mixture thereof. The modulator may be an up-modulator if it increases activity of the enzyme by any mechanism described herein, for example by acting as an enzyme stimulator, enzyme agonist, stabilizer of enzyme (e.g. by inhibiting degradation), activator of transcription of gene encoding the enzyme, activator of translation of the mRNA of the gene encoding the enzyme or a mixture thereof. The terms “modulator” refer to a down-regulation or up-regulation of enzymatic activity. In some embodiments, modulation may refers to a reduction of about 20%, 30%, 40%, 50%, 60%, 70%, 80%, 90% or 95% of the targeted activity or a increase by about 20%, 40%, 60%, 80%, 100%, 200%, 500%, 1000% or 5000%. The reduction or the increase may be a consequence if change in amount of protein, as a consequence of altered protein expression or degradation, or inhibition of enzyme activity by competitive, noncompetitive or uncompetitive mechanism, or activation by allosteric mechanism or a combination thereof.

The terms “inhibitor,” “down-modulator” refer to a down-regulation of enzymatic activity. In some embodiments, inhibition may refers to a reduction of about 200%, 30%, 40%, 50%, 60%, 70%, 80%, 90% or 95% of the targeted activity. The reduction may be a consequence if decrease in amount of protein, as a consequence of reduced protein expression or degradation, or inhibition of enzyme activity by competitive, noncompetitive or uncompetitive mechanism or a combination thereof.

The terms “activator,” “up-modulator” refer to a up-regulation of enzymatic activity. In some embodiments, activation may refers to a increase by about 20%, 30%, 40%, 50%, 60%, 700%, 80%, 90%, 100%, 200%, 500%, 1000% or 5000% of the targeted activity. The increase may be a consequence if increase in amount of protein, as a consequence of increased protein expression or reduced degradation, or activation of enzyme activity by allosteric mechanism or a combination thereof.

Although phosphoinositol phospholipids are minor components of cell membranes, they play important roles in cellular signaling pathways. Amongst their many functions, they play role in the IP3 DAG pathway. Their other functions include docking of specific domains of certain proteins that promote the recruitment of additional proteins to the plasma membrane and subsequent activation of signaling cascades. Activation of kinases like AKT, PDPK1 and Btk1 involve docking on the phosphoinositol phospholipids. Some sodium and potassium channels also require phosphoinositol phospholipids for their functions.

Some cancers have lower local levels of PI(4,5)P2 or rapidly turn over their PI(4,5)p2 as compared to normal cells. It was surprisingly found that the front of a moving cell features low levels of PI(4,5)P2 because of localization of the enzymes that metabolize PI(4,5)P2. In contrast, the tumor suppressor PTEN is localized at the back end of the cell, where the levels of PI(4,5)P2 are elevated. Low PI(4,5)P2 levels in these cancers correlate with Arp2/3-mediated F-actin polymerization, which in turn results in more leading edge projections in cells. These kinds of projections and protrusions are found in invadopodia, which play an important role in cancer invasion. Sharma et al., Tks5 and SHIP2 regulate invadopodium maturation, but not initiation, in breast carcinoma cells, Current Biology 23(21):2079-2089, 2013. Therefore, low levels of PI(4,5)P2 are critical for cell polarity during migration and are associated with invasive, migratory and metastatic phenotype. In some aspects, this disclosure relates to a method of treating cancer by administering an effective amount of an up modulating agent that raises PI(4,5)P2 levels, for example, restores the PI(4,5)P2 levels to approximately normal levels. In some embodiments, the administering of an effective amount of an up-modulating agent of PI(4,5)P2 decreases the invasiveness, migration and/or metastasis of cancer cells.

When cells have higher or induced ROCK activity, RhoA is upregulated in the back of the cell. This in turn promotes frontness and leading to an increase in PI3K activity in the front of the cell. Microtubules are thought to promote Rac activity at the leading edge, which requires PI3K activity. However, when microtubule function is blocked using agents like taxol, vinca alcaloids or nocodazole, backness increases. Increasing backness, sets up a feedback loops that promote frontness. These phenomena are spatially regulated and happen at opposite sides of the cell. A similar phenomenon is observed during cytokinesis, where the cells have one middle, which resembles the “back” of a migrating cell, and two “fronts” at the poles. The receptors that control the regulation of all of these enzymes when cells are in a chemical gradient make use of the cellular machinery that evolved to regulate cell division. As disclosed herein, directed migration evolved using the enzymes that were previously controlled by internal cues that regulate cell shape changes during cell division. Similarly, almost every time cellular receptors are activated, it leads to localized lower levels of PI(4,5)P2.

It was further surprisingly found although mutations that lowered PI(4,5)P2 level, such as PTEN, PLCD1 (encodes an isoform of phosphatidylinositol phospholipase C), activating mutations in class IA PI 3-kinase p110a of phosphatidylinositol-4,5-bisphosphate 3-kinase are common in cancers, two simultaneous mutations in cancer that both lower PI(4,5)P2 levels are rare, even though based on frequencies of single mutations, finding a double mutations should be readily created through continual mutations and genomic arrangements in cancers. For instance, there are no known cancers that contain mutations in KRas and PTEN. In some alternative aspects, this invention provides a method of treating cancer by administering targeted therapy that further lower PI(4,5)P2 level in cancers which have lower levels of PI(4,5)P2 compared to normal cells. Administering targeted therapy that further lower PI(4,5)P2 level make the cells very excitable and should prevent their division, inhibiting their propagation and leading to death. In some aspects, this invention provides a method of treating cancer by administering targeted therapy that lower PI(4,5)P2 level.

In some embodiments, this disclosure relates to a method of treating cancer by administering an effective amount of an up modulating agent that raises PI(4,5)P2 levels, for example, restores the PI(4,5)P2 levels to approximately normal levels. In some embodiments, the administering of an effective amount of an up-modulating agent of PI(4,5)P2 decreases the invasiveness, migration and/or metastasis of cancer cells and can be used to treat the cancer, or to prevent or treat metastasis. In some aspects, cancers with lower levels of PI(4,5)P2 or that are turning over their PI(4,5)P2 levels more rapidly than normal cells may be treated with agents that increase the levels of PI(4,5)P2 (up modulating agents). Any upregulating agent as disclosed herein may be used in these methods.

In some embodiments, this disclosure provides a method of treating cancer in a subject comprising administering a therapeutically effective amount of an agent that modulates the level of PI(4,5)P2 in a cancer cell of the subject. In some embodiments, this invention provides a method of treating cancer by administering a therapeutically effective amount of a modulating agent that lowers the PI(4,5)P2 level in a subject in need thereof. Any downregulating agent as disclosed herein may be used in these methods.

In some embodiments, this disclosure relates to modulating the levels of PI(4,5)P2 by modulating one or more enzymes that modulate PI(4,5)P2. In some embodiments, the agent is a modulator of an enzyme selected from the group consisting of a phosphatidylinositol 4 phosphate 5 kinase, a phosphatidylinositol 5 phosphate 4 kinase, a phosphatidylinositol-3,4,5-trisphosphate 3-phosphatase (the tumor suppressor PTEN), a phosphatidylinositol-4,5-bisphosphate 3-kinase, a SH2-containing inositol phosphatase (SHIP) and a phosphatidylinositol phospholipase C. In some aspects the PI(4,5)P2 modulating agent may be an agent that increases or decreases the transcription, expression, or activity of one or more enzymes that produce PI(4,5)P2 or one or more enzymes that use PI(4,5)P2 as a substrate.

In some embodiments, this disclosure relates to modulating the levels of PI(4,5)P2 by modulating one or more enzymes that produce PI(4,5)P2. In some embodiments, this disclosure relates to modulating the levels of PI(4,5)P2 by modulating one or more enzymes that produce PI(4,5)P2, for example, a phosphatidylinositol 4 phosphate 5 kinase, a phosphatidylinositol 5 phosphate 4 kinase, and/or a phosphatidylinositol-3,4,5-trisphosphate 3-phosphatase (the tumor suppressor PTEN).

In some embodiments, this disclosure relates to up-modulating the levels of PI(4,5)P2 by administering to a subject in need thereof an effective amount of a modulator that increases the activity level or amount of one or more enzymes that produce PI(4,5)P2, thereby upregulating PI(4,5)P2. In some embodiments an up modulator of an enzyme that produces PI(4,5)P2 is selected from the group consisting of stimulator or agonist. In some aspects the PI(4,5)P2 up-modulating agent may be an agent that increases the transcription, expression, or activity of one or more enzymes that produce PI(4,5)P2.

In other embodiments, this disclosure relates to down-modulating the levels of PI(4,5)P2 by administering to a subject in need thereof an effective amount of a modulator that decreases the activity level or amount of one or more enzymes that produce PI(4,5)P2, thereby downregulating PI(4,5)P2. In some embodiments a down-modulator of an enzyme that produces PI(4,5)P2 is selected from the group consisting of an antagonist, blocker, inhibitor or other downregulator of one or more enzymes that produce PI(4,5)P2. In some embodiments the PI(4,5)P2 down-modulating agent may be an agent that decreases the transcription, expression, or activity of one or more enzymes that produce PI(4,5)P2.

In some embodiments, this disclosure relates to modulating the levels of PI(4,5)P2 by modulating one or more enzymes that use PI(4,5)P2 as a substrate. In some embodiments, this disclosure relates to modulating the levels of PI(4,5)P2 by modulating one or more enzymes that metabolize PI(4,5)P2, for example, a phosphatidylinositol-4,5-bisphosphate 3-kinase, a SH2-containing inositol phosphatase (SHIP) and/or a phosphatidylinositol phospholipase C. In some embodiments, this disclosure relates to modulating the levels of PI(4,5)P2 by administering to a subject in need thereof a therapeutically effective amount of a modulator of one or more enzymes that metabolize PI(4,5)P2. In some aspects the modulator of the enzymes that metabolize PI(4,5)P2 is selected from the group consisting of stimulator, agonist, antagonist and inhibitor. In some aspects the modulator of the enzymes that metabolize PI(4,5)P2 may be an agent that increases or decreases the transcription, expression, or activity of one or more enzymes that metabolize PI(4,5)P2.

In some aspects, this disclosure relates to down modulating the levels of PI(4,5)P2 by up regulating or up modulating, e.g., stimulating or activating one or more enzymes that metabolize PI(4,5)P. In other aspects, this disclosure relates to up modulating the levels of PI(4,5)P2 by down regulating, e.g., blocking, inhibiting and/or antagonizing, one or more enzymes that metabolize PI(4,5)P2. In some aspects the PI(4,5)P2 down-modulating agent may be an agent that increases the transcription, expression, or activity of one or more enzymes that metabolize PI(4,5)P2.

Because of central importance of PI(4,5)P2, it is constantly metabolized by several enzymes. These enzymes include phosphatidylinositol 4 phosphate 5 kinase, phosphatidylinositol 5 phosphate 4 kinase, phosphatidylinositol-3,4,5-trisphosphate 3-phosphatase, phosphatidylinositol-4,5-bisphosphate 3-kinase SH2-containing inositol phosphatase (SHIP), and phosphoinositide phospholipase C.

Phosphatidylinositol 4 phosphate 5 kinases (EC 2.7.1.68). These enzymes generate PI(4,5)P2 by catalysis of the following biochemical reaction:

ATP+1-phosphatidyl-1D-myo-inositol 4-phosphate [symbol] ADP+1-phosphatidyl-1D-myo-inositol 4,5-bisphosphate. The systematic name of this enzyme is ATP:1-phosphatidyl-1D-myo-inositol-4-phosphate 5-phosphotransferase. Other commonly-used names include diphosphoinositide kinase, PIP kinase, phosphatidylinositol 4-phosphate kinase, phosphatidylinositol-4-phosphate 5-kinase, and type I PIP kinase. This enzyme can also phosphorylate the 4-position of phosphatidylinositol 3-phosphate, and 5-position of phosphatidylinositol, phosphatidylinositol 3-phosphate and phosphatidylinositol 3,4-bisphosphate. The activity of phosphatidylinositol 4 phosphate 5 kinases may be modulated using a chemical inhibitor, a chemical modulator, a chemical stimulator, a polynucleotide-based agent affecting transcription or translation of the phosphatidylinositol 4 phosphate 5 kinase and a mixture thereof. Inhibitors of phosphatidylinositol 4 phosphate 5 kinases that can be used in methods of the current invention include but are not limited to 2′(3′)-O-(2,4,6-Trinitrophenyl)ATP (Kwok et al. Prep Biochem Biotechnol. 26(1):1-19, 1996), quercetin (Urumow and Wieland, Biochim Biophys Acta 1052(1):152-8, 1990.). Gamma S-pppG is an activator of Phosphatidylinositol 4-phosphate (PIP) kinase (E.C. 2.7.1.68) (Urumow and Wieland, Biochim Biophys Acta 1052(1):152-8, 1990).

Phosphatidylinositol 5 phosphate 4 kinases (EC 2.7.1.149): These enzymes generate PI(4,5)P2 by catalysis of the following biochemical reaction:

ATP+1-phosphatidyl-1D-myo-inositol 5-phosphate [symbol] ADP+1-phosphatidyl-1D-myo-inositol 4,5-bisphosphate.

The systematic name of this enzyme is ATP:1-phosphatidyl-1D-myo-inositol-5-phosphate 4-phosphotransferase. Other commonly-used names are type H PIP kinase and 1-phosphatidylinositol-5-phosphate 4-kinase. The activity of phosphatidylinositol 5 phosphate 4 kinases may be modulated using a chemical inhibitor, a chemical modulator, a chemical stimulator, a polynucleotide-based agent affecting transcription or translation of the phosphatidylinositol 4 phosphate 5 kinase and a mixture thereof. Inhibitors of phosphatidylinositol 4 phosphate 5 kinases, that can be used in methods of the current invention include but are not limited to SAR088 (Voss et al. Biochem Biophys Res Commun. 449(3):327-31, 2014), tyrphostin, (2E)-2-(3,4-dihydroxybenzoyl)-3-(3,4-dihydroxyphenyl)prop-2-enenitrile, (2E)-2-(3,4-dihydroxybenzoyl)-3-(4-hydroxy-3-iodo-5-methoxyphenyl)prop-2-enenitrile, (3E)-5-amino-3-[(2Z)-1-cyano-2-(3H-indol-3-ylidene)ethylidene]-2,3-dihydro-1H-pyrazole-4-carbonitrile, (3Z)-2-amino-4-(3,4,5-trihydroxyphenyl)buta-1,3-diene-1,1,3-tricarbonitrile (Davis et al., PLoS ONE 8, e54127, 2013).

Phosphatidylinositol-4,5-bisphosphate 3-kinases (EC 2.7.1.153): These enzymes phosphorylate phosphatidylinositol-4,5-bisphosphate, and convert it to phosphatidylinositol-3, 4,5-trisphosphate by catalysis of the following reaction:

ATP+1-phosphatidyl-1D-myo-inositol 4,5-bisphosphate [symbol] ADP+1-phosphatidyl-1D-myo-inositol 3,4,5-trisphosphate

The systematic name of this enzyme class is ATP:1-phosphatidyl-1D-myo-inositol-4,5-bisphosphate 3-phosphotransferase. This enzyme is also called type I phosphoinositide 3-kinase. This enzyme plays role in a number of pathways that include: inositol phosphate metabolism, ErbB signaling pathway, phosphatidylinositol signaling system, mTOR signaling pathway, apoptosis, VEGF signaling pathway, focal adhesion, Toll-like receptor signaling pathway, JAK-STAT signaling pathway, natural killer cell mediated cytotoxicity, T cell receptor signaling pathway, B cell receptor signaling pathway, Fc epsilon RI signaling pathway, leukocyte transendothelial migration, regulation of actin cytoskeleton, insulin signaling pathway, and progesterone-mediated oocyte maturation. Phosphatidylinositol-4,5-bisphosphate 3-kinases play roles in pathogenesis of Type II diabetes mellitus, colorectal cancer, renal cell carcinoma, pancreatic cancer, endometrial cancer, glioma, prostate cancer, melanoma, chronic myeloid leukemia, acute myeloid leukemia, small cell lung cancer, and non-small cell lung cancer.

Many inhibitors of phosphatidylinositol-4,5-bisphosphate 3-kinases are known and can be used in methods of the current invention. These inhibitors include but are not limited to AEZS-136, BAY 80-6946, BEZ235, BKM120, CAL263, CUDC-907, demethoxyviridin, GNE-477, GSK1059615, IC87114, idelalisib, INK1117, IPI-145, LY29400, Palomid 529, perifosine, PI-103, PWT33597, PX-866, RP6503, RP6530, SF1126, TG100-115, TGR 1202, wortmannin, XL147 (SAR245408), XL765 (SAR245409), ZSTK474 a polynucleotide-based agent affecting transcription or translation of the phosphatidylinositol-4,5-bisphosphate 3-kinase.

Phosphoinositide phospholipase C (EC 3.1.4.11): These enzymes cleave the bond between diacylglycerol and phosphoinocitide in a reaction represented as follows:

1-phosphatidyl-1D-myo-inositol 4,5-bisphosphate+H₂O [symbol] 1D-myo-inositol 1,4,5-trisphosphate+diacylglycerol

Phosphoinositide phospholipase C is also known as 1-phosphatidyl-D-myo-inositol-4,5-bisphosphate inositoltrisphosphohydrolase, 1-phosphatidylinositol-4,5-bisphosphate phosphodiesterase, monophosphatidylinositol phosphodiesterase, phosphatidylinositol phospholipase C, phosphoinositidase C, PI-PLC and triphosphoinositide phosphodiesterase.

Six sub-families of phosphoinositide phospholipase C consisting of a total of 13 separate isoforms that differ in their mode of activation, expression levels, catalytic regulation, cellular localization, membrane binding avidity and tissue distribution. All isoforms hydrolyze phosphatidylinositol-4,5-bisphosphate into inositol trisphosphate and diacylglycerol, the two important second messenger molecules. These molecules then modulate the activity of downstream proteins important for cellular signaling. Inositol trisphosphate is soluble, and diffuses through the cytoplasm and interacts with its receptors on the endoplasmic reticulum, causing the release of calcium and raising the level of intracellular calcium. Examples of proteins activated by phosphatidylinositol 34,5-trisphosphate are AKT, PDPK1, Btk1. Diacylglycerol remains tethered to the inner leaflet of the plasma membrane due to its hydrophobic character, and activates protein kinase C (PKC). This results in a host of cellular responses like proliferation, differentiation, apoptosis, cytoskeleton remodeling, vesicular trafficking, ion channel conductance, endocrine function and neurotransmission.

Inhibitors of phosphoinositide phospholipase C that can be used in methods of the current invention include but are not limited to is selected from the group consisting of D609 (Tricyclodecan-9-yl-xanthogenate), edelfosine, U73122, a polynucleotide-based agent affecting transcription or translation of the phosphoinositide phospholipase C and a mixture thereof.

SH2-containing inositol phosphatase (SHIP) (EC 3.1.3.36): These enzymes dephosphorylate PI(4,5)P2 in a reaction represented as follows:

1-phosphatidyl-1D-myo-inositol 4,5-bisphosphate+H2O [symbol] 1-phosphatidyl-1D-myo-inositol 4-phosphate+phosphate

The systematic name of this enzyme class is phosphatidyl-myo-inositol-4,5-bisphosphate 4-phosphohydrolase. Other names in common use include type II inositol polyphosphate 5-phosphatase, triphosphoinositide phosphatase, IP3 phosphatase, PtdIns(4,5)P2 phosphatase, triphosphoinositide phosphomonoesterase, diphosphoinositide phosphatase, inositol 1,4,5-triphosphate 5-phosphomonoesterase, inositol triphosphate 5-phosphomonoesterase, phosphatidylinositol-bisphosphatase, phosphatidyl-myo-inositol-4,5-bisphosphate phosphatase, phosphatidylinositol 4,5-bisphosphate phosphatase, polyphosphoinositol lipid 5-phosphatase, and phosphatidyl-inositol-bisphosphate phosphatase.

Inhibitors of phosphoinositide phospholipase C that can be used in methods of the current invention include but are not limited to is selected from the group consisting of 3-benzyl-oxybenzene 1,2,4-trisphosphate, 3-hydroxybenzene 1,2,4-trisphosphate, benzene 1,2,3,4-tetrakisphosphate, benzene 1,2,3-trisphosphate, benzene 1,2,4,5-tetrakisphosphate, benzene 1,2,4-trisphosphate, benzene 1,3,5-trisphosphate, biphenyl 2,3′,4,5′,6-pentakisphosphate (Mills et al. ChemBioChem 9, 1757-1766, 2008).

Phosphatidylinositol-3,4,5-trisphosphate 3-phosphatase (EC 3.1.3.67): This enzyme generates PI(4,5)P2 from phosphatidylinositol-3,4,5-trisphosphate catalyzing the following reaction:

Phosphatidylinositol 3,4,5-trisphosphate+H₂O [symbol] phosphatidylinositol 4,5-bisphosphate+phosphate

The systematic name of this enzyme class is 1-phosphatidyl-1D-myo-inositol-3,4,5-trisphosphate 3-phosphohydrolase. Other names include PTEN, MMAC1, and phosphatidylinositol-3,4,5-trisphosphate 3-phosphohydrolase. This enzyme participates in several metabolic pathways including inositol phosphate metabolism, phosphatidylinositol signaling system, p53 signaling pathway, focal adhesion, tight junction. As a consequence, this enzyme plays a role in pathogenesis of several cancers like endometrial cancer, glioma, prostate cancer, melanoma, and small cell lung cancer.

Phosphatidylinositol-3,4,5-trisphosphate 3-phosphatase is the tumor suppressor PTEN (phosphatase and tensin homolog). PTEN mutations are associated with many types of cancers. PTEN is a tumor suppressor protein involved in the regulation of the cell cycle, preventing cells from growing and dividing too rapidly, through negative regulation of Akt/PKB signaling pathway.

Inhibitors of phosphatidylinositol-3,4,5-trisphosphate 3-phosphatase that can be used in methods of the current invention include but are not limited to oncomiR, MIRN21, SF1670, a polynucleotide-based agent affecting transcription or translation of the phosphatidylinositol-3,4,5-trisphosphate 3-phosphatase and a mixture thereof.

The methods of current invention may involve administration of one or more compounds listed above, or their pharmacologically salts, hydrates, polymorphs, along with one or more pharmaceutically-acceptable excipients, carriers, or diluents. Pharmaceutically-acceptable excipients, carriers, or diluents of the invention include but are not limited to sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

In some aspects, the methods of treatment described herein further comprises screening a cancer patient to identify a suitable treatment, the method comprising (i) providing a sample from the subject and a control sample; and (ii) testing the sample to detect one or more of (a) level of phosphatidylinositol-4,5-bisphosphate, (b) mutation in a gene encoding an enzyme modulating PI(4,5)P2; and (c) activity of an enzyme selected from the group consisting of the enzymes modulating PI(4,5)P2.

In some aspects, the sample may comprise cancer cells or a fraction thereof such as membrane fraction or isolated lipids used to determine level of PI(4,5)P2. In some aspects, the sample may be a body fluid used for detecting the level of PI(4,5)P2 released from cells. In some embodiments, sample from the subject used in the sample from the subject is selected from the group consisting of blood, serum, plasma, lymph, urine, saliva, a mucosal secretion, a vaginal secretion, cerebrospinal fluid, serosal fluid, ascites fluid, pleural fluid, pericardial fluid, peritoneal fluid, abdominal fluid, lavage fluid, fecal matter, sputum, biopsy sample, autopsy sample, tears, washings obtained during a medical procedure. The sample may also be a processed sample, for example, extracted, isolated or purified lipids, DNA, protein, nucleic acids, amino acids, metabolites, analytes, and/or conditioned culture medium used for cultivating at least one cell from a subject and a cell lysate. In some embodiments, the control sample may be sample derived from normal tissue from the subject, a biological sample derived from a normal subject or a solution comprising phosphatidylinositol-4,5-bisphosphate.

In some embodiments, levels of PI(4,5)P2 are measured by techniques including but not limited to ELISA, FACS, immunocytochemistry, radioimmunoassay, immunofluorescence microscopy and western blotting.

In some embodiments, levels of PI(4,5)P2 are measured by forming a first complex comprising PI(4,5)P2 and a binding agent. In some embodiments, the method of measuring levels of PI(4,5)P2 comprises contacting a sample derived from a subject with the binding agent, including but not limited to a pleckstrin homology domain specific for PI(4,5)P2 (Holz et al The Journal of Biological Chemistry, 275:17878-17885, 2000), a polyclonal antibody, a monoclonal antibody, a bispecific antibody, a chimeric antibody, a humanized antibody, a single chain antibody, aptamer and a binding fragment of the polyclonal, monoclonal, bispecific, chimeric, humanized, or single chain antibody. Some monoclonal antibodies suitable for the methods described herein include but are not limited to anti-PIP2 antibody clones 2C11, KT-10 and AM-212. In some aspects, the binding agents are conjugated to one or more detectable agent including but not limited to an enzyme, a fluorescent label, a chemiluminescent label, a nanoparticle label, a radioactive isotope, biotin, avidin and streptavidin.

Binding agents for PI(4,5)P2 may also be used in some instances as therapeutic modulating agents to downregulate the effective levels of PI(4,5)P2, according to the methods described herein.

In some embodiments, level of PI(4,5)P2 are measured by forming a second complex between PI(4,5)P2, a binding agent and a second binding agent, the method comprising, contacting a first complex comprising PI(4,5)P2 and a binding agent with a second binding agent, wherein the second binding agent includes but is not limited to anti-pleckstrin homology domain antibody, a secondary antibody against an antibody, a lectin, and vitamin B12.

In some embodiments, levels of PI(4,5)P2 are measured by an enzymatic assay for an enzyme PI(4,5)P2 as a substrate. In some embodiments, levels of PI(4,5)P2 are measured by an enzymatic assay for phosphatidylinositol-4,5-bisphosphate 3-kinases, wherein the change levels of ATP, the second substrate of the reaction are measured to determine the levels of PI(4,5)P2. In some embodiments, the changes in levels of ATP are measured on the basis of light emission following a secondary reaction using luciferase. In some embodiments, the changes in levels of ATP are measured using a coupled pyruvate kinase and lactate dehydrogenase reactions that use phosphoenol pyruvate and NADH, and changes in levels of PI(4,5)P2 are quantitatively correlated with oxidation of NADH to NAD⁺, and change absorbance at 340 nm.

In some aspects the levels of PI(4,5)P2 are measured by a chromatography-based assay, including but not limited to thin layer chromatography and HPLC. In some embodiments, the levels of PI(4,5)P2 are measured by a mass spectroscopy-based assay.

In some embodiments, the method of diagnosis of cancer in a subject comprises:
(a) providing a sample from a subject and a control sample;
(b) extracting lipids from the sample;
(c) contacting the extracted lipid with a methylating agent for time and conditions sufficient to methylate PI(4,5)P2 in the extracted lipid;
(d) separating the methylated PI(4,5)P2 using chromatography;
(e) infusing into a mass spectrometer in positive ion mode; and
(f) detecting the methylated PI(4,5)P2.

In some embodiments, the methylating agent includes but is not limited to TMS-diazomethane, bromomethane, diazomethane, 2,2-dimethoxypropane, dimethyl carbonate, dimethyl dicarbonate, dimethyl sulfate, 1,2-dimethylhydrazine, dimethylzinc, eschweiler-clarke reagent, methyl fluorosulfonate, methylcobalamin, methyl iodide, iodomethane, methyl methanesulfonate, methyl triflate, methyl trifluoromethansulfonate and trimethyloxonium tetrafluoroborate. In some embodiments, the chromatography may be HPLC or HTLC. In some embodiments, alternatively, solid phase extraction may also be used in place of chromatography. In some aspects, mutations in a gene encoding an enzyme modulating PI(4,5)P2 are determined using sequencing-based assays.

In some aspects, levels of enzymes modulating PI(4,5)P2 are measured by techniques including but not limited to ELISA, FACS, immunocytochemistry, radioimmunoassay, immunofluorescence microscopy and western blotting. In some aspects, activity of an enzyme modulating PI(4,5)P2 are measured by contacting a sample derived from a subject with substrates of the reactions the enzymes catalyze and measuring the products directly or using coupled reactions. In some embodiments, the activity of phosphatidylinositol-4,5-bisphosphate 3-kinases are measured contacting a sample derived from a subject with PI(4,5)P2 and ATP and change levels of ATP are measured coupled reactions like luciferase or pyruvate kinase-lactate dehydrogenase, and measuring light emission or change in absorbance at 340 nm.

In some embodiments, when the level of phosphatidylinositol-4,5-bisphosphate in a sample derived from the subject is low or rapidly turning over, the method of treatment of cancer in a subject comprises administration of therapeutically sufficient amount of an inhibitor of an enzyme that metabolizes PI(4,5)P2 or an activator of an enzyme that produces PI(4,5)P2, to restore the levels of PI(4,5)P2 and stops leading edge projections in cells, associated with invasiveness, migration and metastasis. In some alternative embodiments, the method of treatment of cancer in a subject, when the level of phosphatidylinositol-4,5-bisphosphate in a sample derived from the subject is low, comprises administration of therapeutically sufficient amount of an activator of an enzyme that metabolizes PI(4,5)P2 or an inhibitor of an enzyme that produces PI(4,5)P2, to inhibit cancer cell diviion and make them very excitable.

In some embodiments, when the level of PI(4,5)P2 in a sample from a subject is low or rapidly turning over, the agent used for treating cancer in the subject is an agent that modulates the enzymes that produce or metabolize PI(4,5)P2. In some embodiments, when the level of PI(4,5)P2 in a sample from a subject is low, the agent used for treating cancer in the subject is a modulator of the enzymes that produce PI(4,5)P2. In some embodiments, when the level of PI(4,5)P2 in a sample from a subject is low, the agent used for treating cancer in the subject is a modulator of the enzymes that metabolize PI(4,5)P2.

In some asp embodiments, when the level of PI(4,5)P2 in a sample from a subject is low or rapidly turning over, the agent used for treating cancer in the subject is an agent that increases the level of PI(4,5)P2. In some embodiments, the agent used for treating cancer in the subject is a stimulator or an agonist of enzymes that produce PI(4,5)P2, or an inhibitor or an antagonist of enzymes that metabolize PI(4,5)P2.

In some alternative embodiments, when the level of PI(4,5)P2 in a sample from a subject is low or rapidly turning over, the agent used for treating cancer in the subject is an agent that further decreases the level of PI(4,5)P2. In some embodiments, the agent used for treating cancer in the subject is an inhibitor or an antagonist of enzymes that produce PI(4,5)P2, or a stimulator or an agonist of enzymes that metabolize PI(4,5)P2.

Some cancers have high PI(4,5)P2 levels and they may be treated by further increasing levels of PI(4,5)P2

In some embodiments, when the level of PI(4,5)P2 in a sample from a subject is high the agent used for treating cancer in the subject is an agent that further increases the level of PI(4,5)P2. In some embodiments, the agent used for treating cancer in the subject is an activator or an agonist of enzymes that produce PI(4,5)P2, or a inhibitor or an antagonist of enzymes that metabolize PI(4,5)P2, or a combination thereof.

In some embodiments, when a subject has a mutation in a gene encoding a phosphatidylinositol 4 phosphate 5 kinase or reduced activity of a phosphatidylinositol 4 phosphate 5 kinase, the agent used for treating cancer in the subject is a down modulator, inhibitor, blocker or antagonist of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some embodiments, when a subject has a mutation in a gene encoding a phosphatidylinositol 4 phosphate 5 kinase or reduced activity of a phosphatidylinositol 4 phosphate 5 kinase, the agent used for treating cancer in the subject is an upregulator, agonist or stimulator of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some embodiments, when a subject has a mutation in a gene encoding a phosphatidylinositol 4 phosphate 5 kinase or reduced activity of a phosphatidylinositol 4 phosphate 5 kinase, the agent used for treating cancer in the subject is a down modulator, inhibitor, blocker or antagonist of one or more of the enzymes that metabolize PI(4,5)P2.

In some alternative embodiments, when a subject has a mutation in a gene encoding a phosphatidylinositol 4 phosphate 5 kinase or reduced activity of a phosphatidylinositol 4 phosphate 5 kinase, the agent used for treating cancer in the subject is an upregulator, agonist or stimulator of one or more of the enzymes that metabolize PI(4,5)P2.

In some embodiments, when a subject has a mutation in a gene encoding a phosphatidylinositol 5 phosphate 4 kinase or reduced activity of a phosphatidylinositol 5 phosphate 4 kinase, the agent used for treating cancer in the subject is a down modulator, inhibitor, blocker or antagonist of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some alternative embodiments, when a subject has a mutation in a gene encoding a phosphatidylinositol 5 phosphate 4 kinase or reduced activity of a phosphatidylinositol 5 phosphate 4 kinase, the agent used for treating cancer in the subject is an upregulator, agonist or stimulator of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some embodiments, when a subject has a mutation in a gene encoding a phosphatidylinositol 5 phosphate 4 kinase or reduced activity of a phosphatidylinositol 5 phosphate 4 kinase, the agent used for treating cancer in the subject is is a down modulator, inhibitor, blocker or antagonist of one or more of the enzymes that use PI(4,5)P2 as a substrate.

In some embodiments, when a subject has a mutation in a gene encoding a phosphatidylinositol 5 phosphate 4 kinase or reduced activity of a phosphatidylinositol 5 phosphate 4 kinase, the agent used for treating cancer in the subject is an upregulator, agonist or stimulator of one or more of the enzymes that use PI(4,5)P2 as a substrate.

In some embodiments, when a subject has a mutation in a gene encoding a phosphatidylinositol-3,4,5-trisphosphate 3-phosphatase or reduced activity of a phosphatidylinositol-3,4,5-trisphosphate 3-phosphatase, is a down modulator, inhibitor, blocker or antagonist of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some alternative embodiments, when a subject has a mutation in a gene encoding a phosphatidylinositol-3,4,5-trisphosphate 3-phosphatase or reduced activity of a phosphatidylinositol-3,4,5-trisphosphate 3-phosphatase, is an upregulator, agonist or stimulator of a phosphatidylinositol 4 phosphate 5 kinase, a phosphatidylinositol 5 phosphate 4 kinase, a phosphatidylinositol-3,4,5-trisphosphate 3-phosphatase or a mixture thereof.

In some embodiments, when a subject has a mutation in a gene encoding a phosphatidylinositol-3,4,5-trisphosphate 3-phosphatase or reduced activity of a phosphatidylinositol-3,4,5-trisphosphate 3-phosphatase, the agent used for treating cancer in the subject is a down modulator, inhibitor, blocker or antagonist of one or more of the enzymes that use PI(4,5)P2 as a substrate.

In some alternative embodiments, when a subject has a mutation in a gene encoding a phosphatidylinositol-3,4,5-trisphosphate 3-phosphatase or reduced activity of a phosphatidylinositol-3,4,5-trisphosphate 3-phosphatase, the agent used for treating cancer in the subject is a upregulator, agonist or stimulator of one or more of the enzymes that use PI(4,5)P2 as a substrate.

Pharmaceutical Formulations, Doses, and Administration:

Pharmaceutical compositions for use in accordance with the present invention may be formulated in a conventional manner using one or more physiologically acceptable carriers or excipients. The formulation may be prepared to provide for rapid or slow release; immediate, delayed, timed, or sustained release; or a combination thereof. Formulations may be in the form of liquids, solutions, suspensions, emulsions, elixirs, syrups, electuaries, mouthwashes, drops, tablets, granules, powders, lozenges, pastilles, capsules, gels, pastes, ointments, creams, lotions, oils, foams, sprays, mists, or aerosols. Formulations may be provided as a patch, adhesive plaster, bandage, dressing, or in the form of depot or reservoir.

The tablet may be formulated for immediate release, sustained release, or delayed or modified release. The tablet may be optionally coated can make the tablet resistant to the stomach acids and it disintegrates in the duodenum, jejunum and colon as a result of enzyme action or alkaline pH. These formulations are known to one of ordinary skill in the art. The tablets may be further coated with sugar, varnish, or wax to mask the taste.

Any suitable concentration of an active pharmaceutical ingredient may be used, where the active pharmaceutical ingredient is administered in an effective amount to achieve its intended purpose. Determination of a therapeutically effective amount for a particular active ingredient is well within the capability of persons skilled in the art.

The therapeutically effective dose of the pharmacologic agent can be administered using any medically acceptable mode of administration. Although the skilled artisan would contemplate any of the modes of administration known to one of ordinary skill, preferably the pharmacologic agent is administered according to the recommended mode of administration, for example, the mode of administration listed on the package insert of a commercially available agent. In general, the dose may comprise 0.01 mg to about 10 g/kg/day.

The compounds described herein may be administered directly, they may also be formulated to include at least one pharmaceutical acceptable carriers, diluents, excipients, adjuvants, fillers, buffers, preservatives, lubricants, solubilizers, surfactants, wetting agents, masking agents, coloring agents, flavoring agents, and sweetening agents. Also, as described herein, such formulation may also include other active agents, for example, other therapeutic or prophylactic agents.

Methods of making a pharmaceutical composition include admixing at least one active compound, as defined above, together with one or more other pharmaceutically acceptable ingredients, such as carriers, diluents, excipients, and the like. When formulated as discrete units, such as tablets or capsules, each unit contains a predetermined amount of the active compound.

Routes of Administration:

In certain embodiments, pharmaceutical compositions of the present invention may be formulated for administration by any route of administration, including but not limited to systemic, peripheral, or topical. Illustrative routes of administration include, but are not limited to, oral, such as by ingestion, buccal, sublingual, transdermal including, such as by a patch, plaster, and the like, transmucosal including, such as by a patch, plaster, and the like, intranasal, such as by nasal spray, ocular, such as by eye drops, pulmonary, such as by inhalation or insufflation therapy using, such as via an aerosol through the mouth or nose, rectal, such as by suppository or enema, vaginal, such as by pessary, parenteral, such as by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and by implant of a depot or reservoir, such as intramuscularly, topical including, such as by cream, gel, ointment, lotion, solution and the like. Dosage of the pharmaceutical compositions may vary by route of administration. Certain administration methods may include the step of administering the composition one or more times a day to obtain the desired therapeutic effect.

Cellular feedback loops lead to unexpected results. For example, raising PI(4,5)P2 levels sets up positive feedback loop that makes the cell more invasive, which leads to increased “backness”. Increasing “backness” can increase “frontness.” Accordingly, the rate turnover of PI(4,5)P2 levels in cancer cells is an informative diagnostic tool for cancer.

PI(4,5)P2 levels may vary in “front” or “back” microenvironments where enzyme activities alter during activation of mobility or feed back loops adjust the PI(4,5)P2 levels. Therefore, a useful method for diagnosis of cancer is determination of the rate of turnover of PI(4,5)P2 levels in cancer cells. The rate of turnover of PI(4,5)P2 levels in cancer cells is altered in many situations. These situations include constitutive activity of an enzyme that uses PI(4,5)P2 as a substrate or low activity of an enzyme that generates PI(4,5)P2 (for example a hypomorphic mutation in PTEN). Accordingly, the cells that are really active, turn over PI(4,5)P2 more rapidly.

In some aspects, the method of diagnosis of cancer in a subject comprises determination of the rate of turnover of PI(4,5)P2 levels in cancer cells.

In some embodiments, the method of diagnosis of cancer in a subject comprises:
(a) contacting a sample comprising at least one cancer cell with labelled PI(4,5)P2 at an initial concentration;
(b) incubating the sample under cell growth conditions for an amount of time sufficient to cause turnover of the PI(4,5)P2;
(c) performing an assay to detect the level of one or more of the PI(4,5)P2, phosphatidylinositol-3,4,5-trisphosphate and inositol 1,4,5-trisphosphate;
(d) comparing the level of PI(4,5)P2, phosphatidylinositol-3,4,5-trisphosphate and inositol 1,4,5-trisphosphate, carrying the label, with the initial concentration; (e) determining the rate of turnover of PI(4,5)P2.

If the rate of turnover of PI(4,5)P2 in the sample from the subject exceeds a threshhold rate of turnover, the subject may have metastatic cancer or be at risk for metastasis.

In some embodiments, the method of diagnosis of cancer in a subject comprises:
(a) contacting a sample from the subject comprising at least one cancer cell with labelled PI(4,5)P2 at an initial concentration;
(b) contacting a control sample comprising at least one cell with labeled PI(4,5)P2;
(b) incubating the sample from the subject and the control sample under growth conditions for an amount of time sufficient to cause turnover of the PI(4,5)P2;
(c) extracting a fraction containing one or more of PI(4,5)P2, phosphatidylinositol-3,4,5-trisphosphate and inositol 1,4,5-trisphosphate;
(d) performing an assay to detect the level of one or more of the PI(4,5)P2, phosphatidylinositol-3,4,5-trisphosphate and inositol 1,4,5-trisphosphate, carrying the label;
(e) comparing the level of the phosphatidylinositol-4,5-bisphosphate, phosphatidylinositol-3,4,5-trisphosphate and inositol 1,4,5-trisphosphate in the sample from the subject with the initial concentration;
(f) comparing the level of the phosphatidylinositol-4,5-bisphosphate, phosphatidylinositol-3,4,5-trisphosphate and inositol 1,4,5-trisphosphate in the control sample with the initial concentration;
(f) determining the rate of turnover of PI(4,5)P2 in the sample from the subject and in the control sample.

If the rate of turnover of PI(4,5)P2 in the sample from the subject is greater than the rate of turnover in the control sample, the subject may have metastatic cancer or be at risk for metastasis. In some embodiments, the rate of turnover of PI(4,5)P2 in the sample from the subject may be normalized against the turnover rate in the control sample.

In some embodiments, the method of diagnosis of cancer in a subject comprises:
(a) providing a sample comprising at least one cancer;
(b) extracting a fraction containing plasma membrane or a fraction containing one or more enzymes that metabolize PI(4,5)P2;
contacting the fraction containing plasma membrane or the fraction containing one or more enzymes that metabolize PI(4,5)P2, with a PI(4,5)P2 at an initial concentration, wherein the PI(4,5)P2 is labelled with a detectable marker;
(b) incubating under a reaction condition for an amount of time sufficient to cause turnover of the PI(4,5)P2;
(c) performing an assay to detect the level of one or more of the PI(4,5)P2, phosphatidylinositol-3,4,5-trisphosphate and inositol 1,4,5-trisphosphate;
(d) comparing the level of PI(4,5)P2, phosphatidylinositol-3,4,5-trisphosphate and inositol 1,4,5-trisphosphate, carrying the label, with the initial concentration; (e) determining the rate of turnover of PI(4,5)P2.

If the rate of turnover of PI(4,5)P2 in the sample from the subject exceeds a threshold rate of turnover, the subject may have metastatic cancer or be at risk for metastasis.

In some embodiments, the control sample is a sample that does not contain cancer cells, for example, a sample containing normal cells from the subject, a membrane fraction of normal cell from the subject, an extract of normal cells from the subject, at least one cell from an animal that does not have cancer, a membrane fraction from at least one cell from an animal that does not have cancer, an extract of at least one cell from an animal that does not have cancer or a solution comprising one or more of phosphatidylinositol-4,5-bisphosphate, phosphatidylinositol-3,4,5-trisphosphate and inositol 1,4,5-trisphosphate.

In some embodiments, the detectable marker used for labelling the labelled phosphatidylinositol-4,5-bisphosphate includes but is not limited to radioactivity and a co-valently attached chemical reporter, wherein the reporter includes but is not limited to a fluorescent probe, a chemiluminiscent probe and a biotin.

In some embodiments, the assay used to detect one or more of the phosphatidylinositol-4,5-bisphosphate, phosphatidylinositol-3,4,5-trisphosphate and inositol 1,4,5-trisphosphate includes but is not limited to an immunological assay, a chromatography-based assay, a mass spectroscopy-based assay, an enzymatic assay.

### EXAMPLES

### Example 1

PI(4,5)P2 functions as an intermediate in the IP3/DAG pathway, which is initiated by ligands binding to G protein-coupled receptors activating the Gq alpha subunit. PI(4,5)P2 is a substrate for hydrolysis by phospholipase C (PLC), a membrane-bound enzyme activated through protein receptors such as al adrenergic receptors. PI(4,5)P2 regulates the function of many membrane proteins and ion channels, such as the M-channel. The products of the PLC catalyzation of PIP2 are inositol 1,4,5-trisphosphate (InsP3; IP3) and diacylglycerol (DAG), both of which function as second messengers. In this cascade, DAG remains on the cell membrane and activates the signal cascade by activating protein kinase C (PKC). PKC in turn activates other cytosolic proteins by phosphorylating them. The effect of PKC could be reversed by phosphatases. inositol 1,4,5-trisphosphate enters the cytoplasm and activates inositol 1,4,5-trisphosphate receptors on the smooth endoplasmic reticulum (ER), which opens calcium channels on the smooth ER, allowing mobilization of calcium ions through specific Ca²⁺ channels into the cytosol. Calcium participates in the cascade by activating other proteins.

Class I PI 3-kinases phosphorylate PI(4,5)P2 forming phosphatidylinositol (3,4,5)-trisphosphate (PtdIns(3,4,5)P3). Both phosphatidylinositol 3, 4,5-trisphosphate and PI(4,5)P2 not only act as substrates for enzymes but also serve as docking phospholipids that bind specific domains that promote the recruitment of proteins to the plasma membrane and subsequent activation of signaling cascades.

### Example 2

### Response of Cells to a Uniform Chemical Stimulus

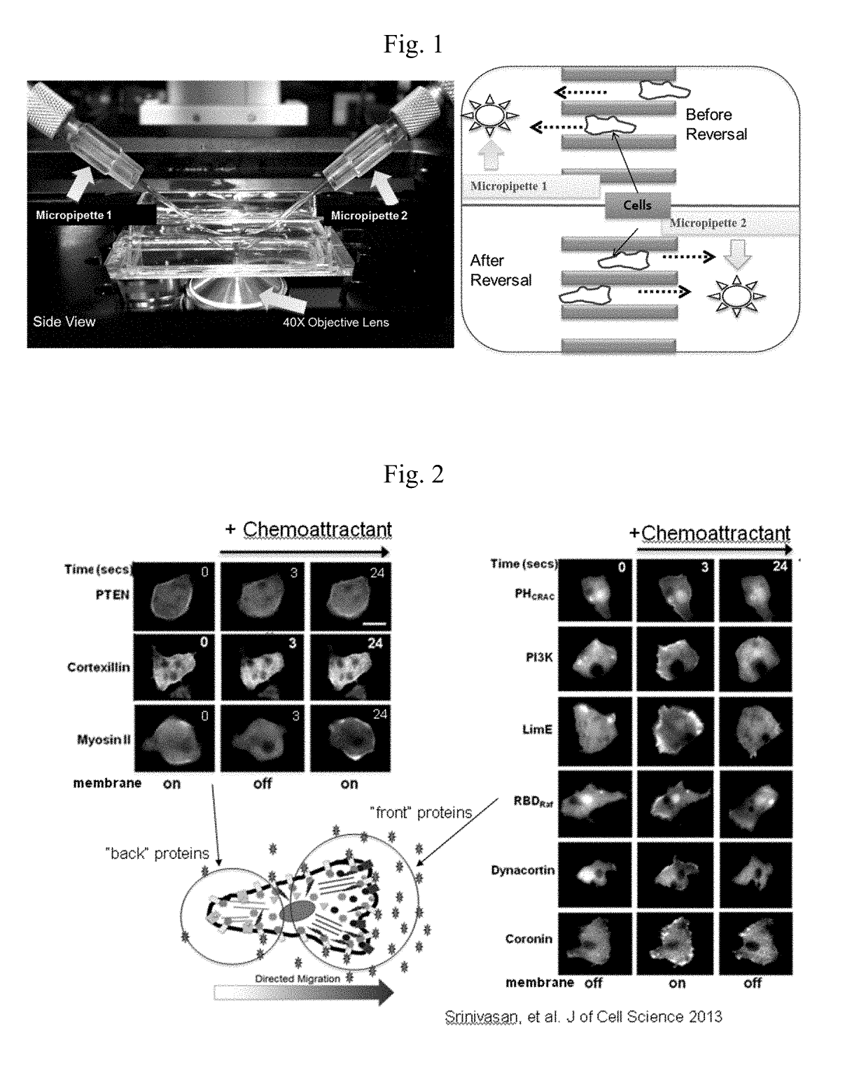

Signaling and cytoskeletal elements were monitored during polarity reversals of migrating Dictyostelium discoideum cells using a novel microfluidic device shown in FIG. 1. Dictyostelium can be made to migrate in the predetermined direction by using chemoattractants like cAMP. folate and lysophosphatidic acid. In the experiments described herein Dictyostelium cells were lured into the PDMS (polydimethylsiloxane) channels by a 1 mM cAMP gradient. In some experiments, after the cells had entered the channel, the gradient was reversed by bringing a second micropipette to the rear of the cells. In these experiments, cells completely broke down their polarity and reestablished it in the opposite direction. The cells used in the experiment expressed GFP-tagged markers cells. Alternatively, the cells were fixed and stained for the indicated markers and observed using fluorescent microscopy.

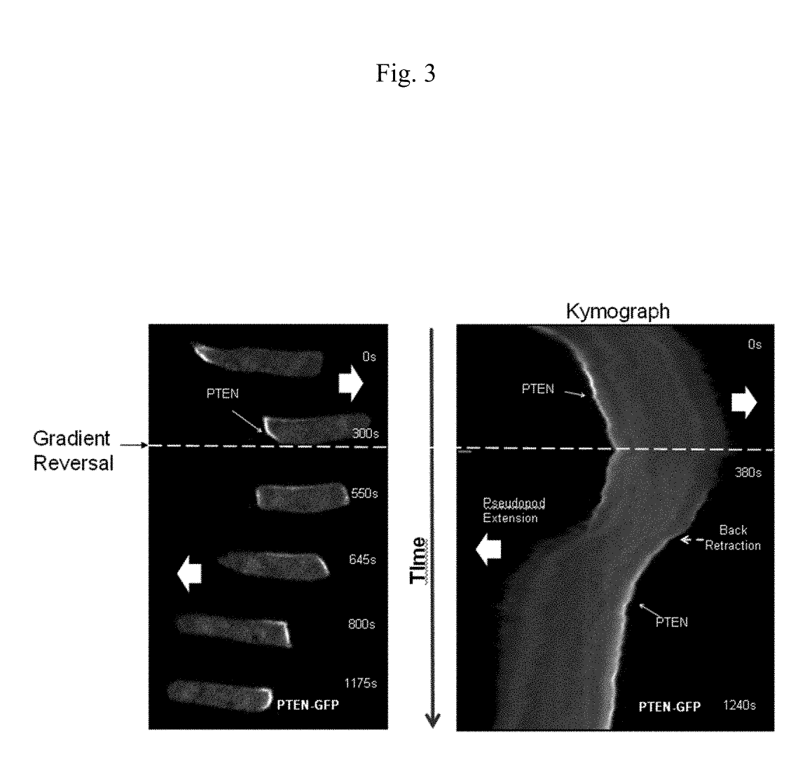

FIG. 2 shows the response of cells to a uniform chemical stimulus. Comparison of the location of GFP tagged molecules before and after uniform addition of 100 piM folic acid, it was found that “backness” and “frontness” in regions of the cell were regulated by plasma membrane (PM) PI(4,5)P2 levels. PI(4,5)P2 3-Kinase (PI3K) and Phospholipase C (PLC) activity reduce PI(4,5)P2 levels at the front, while high PI(4,5)P2 levels contribute to “backness”. PTEN, a protein that produces PI(4,5)P2 from phosphatidylinositol 3,4,5-trisphosphate, moved to back of the cell and PI(4,5)P2 3-Kinase, that uses PI(4,5)P2 as a substrate, moved to front of the cell. The tumor suppressor PTEN catalyses the dephosphorylation of the 3′ phosphate of phosphatidylinositol 3, 4,5-trisphosphate, producing PI(4,5)P2. PTEN contains a PI(4,5)P2 binding motif and helps maintain high PI(4,5)P2 levels at the back of the cell in a positive feedback loop. FIG. 3 shows a time course of movement of PTEN when chemoattractant gradient was reversed.

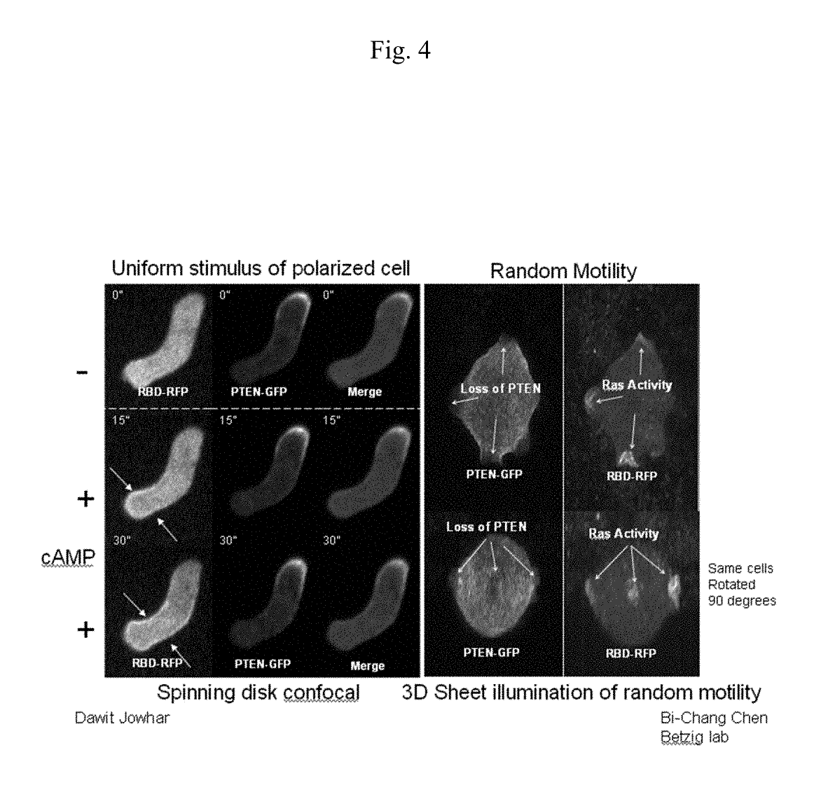

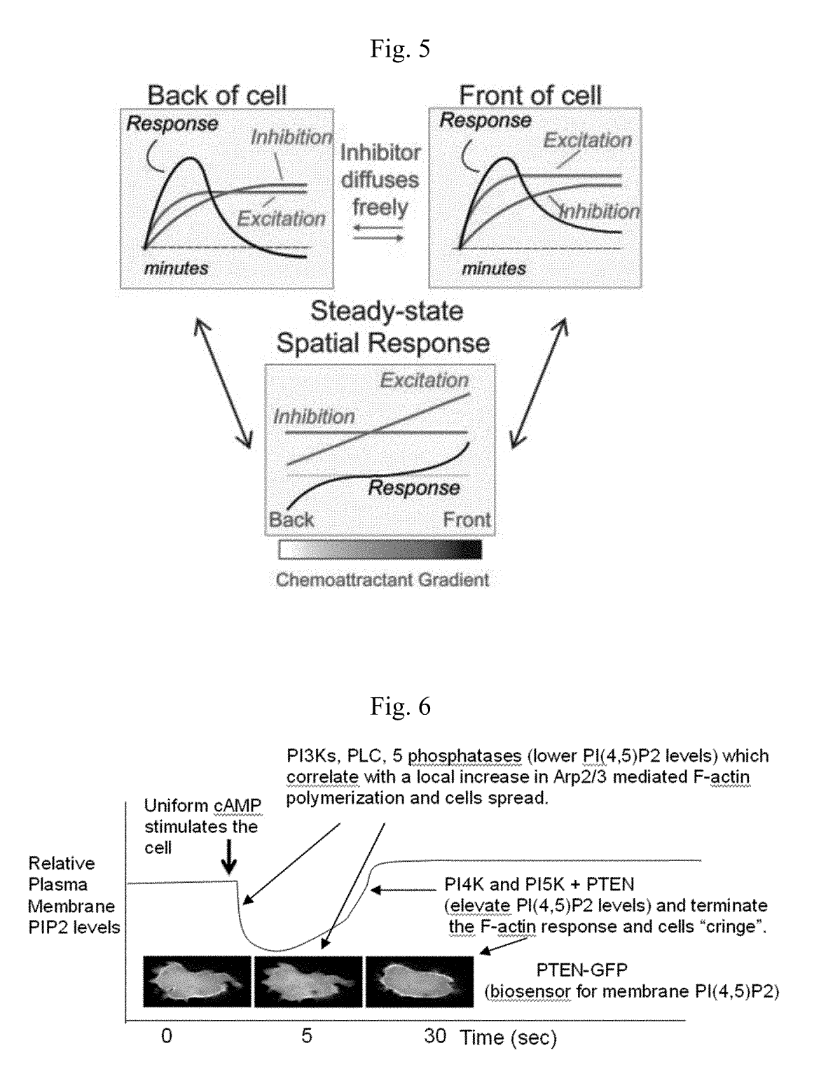

As shown in FIG. 4, interestingly, the activity of the GTPase Ras was reciprocally regulated with local PI(4,5)P2 level during polarity reestablishment, suggesting that high PI(4,5)P2 levels inhibit Ras activity, supporting a negative feedback loop. Cells lacking PLC and treated with PI3K inhibitors are still able to reduce PI(4,5)P2 levels and break symmetry when receptors are activated, suggesting that other 4′- or 5′-phosphatases are activated by chemoattractant and contribute to “frontness”, providing yet another level of redundancy. FIG. 5 shows the Global Inhibition Model (LEGI) that explains cell responses to uniform stimulus and to a chemical gradient.

For measuring PI(4,5)P2, a pleckstrin homology domain specific for PI(4,5)P2, fused to GFP was used. FIG. 6 shows that levels of PI(4,5)P2 drop rapidly in response to a uniform stimulus of cAMP as a number of enzymes that use phosphatidylinositol-4,5-bisphosphate as a substrate are activated. This supports that low levels of phosphatidylinositol-4,5-bisphosphate contribute to the activation of ARP2/3 mediated actin responses. With a slight delay, phosphatidylinositol-4 and phosphatidylinositol 5 kinases are activated and begin to elevate the phosphatidylinositol-4,5-bisphosphate levels. PTEN, which contains a phosphatidylinositol-4,5-bisphosphate binding site is then recruited, which helps lower phosphatidylinositol-3,4,5-triphosphate levels and in a positive feedback loop, further raises phosphatidylinositol-4,5-bisphosphate. This supports that high levels of phosphatidylinositol-4,5-bisphosphate are associated with formin-mediated actomyosin contraction. The elusive “inhibition” in the LEGI model is likely largely controlled by the enzymes that raise phosphatidylinositol-4,5-bisphosphate levels.

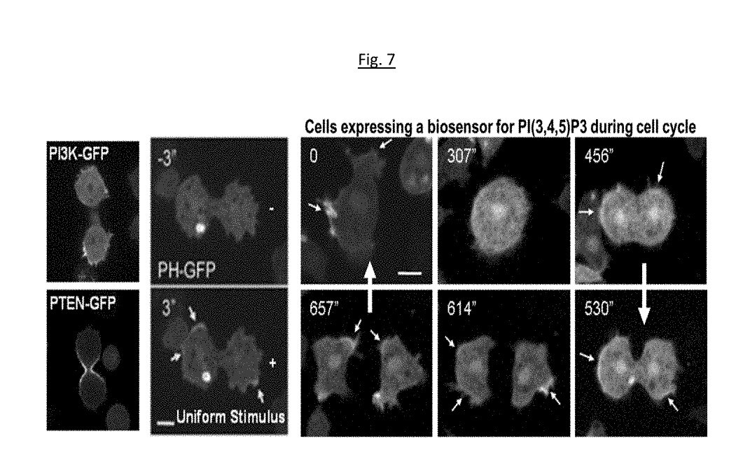

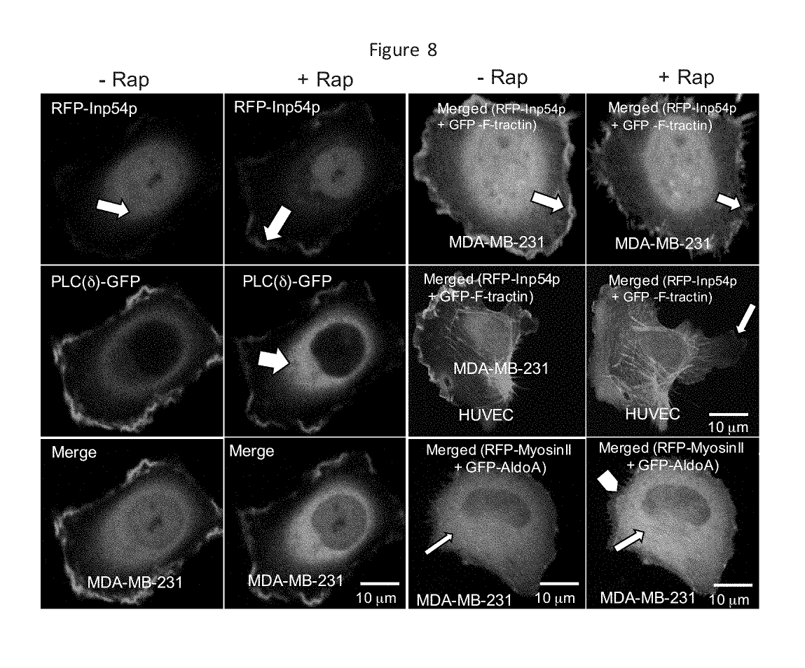

FIG. 7 shows changes in phosphatidylinositol phosphates are similarly regulated during cytokinesis. Localization of phosphatidylinositol-3-kinase and PTEN during anaphase in Dictyostelium (Far left, top and bottom) and cells before (−3) and after (3 sec) stimulation with chemoattractant are shown. Cells expressing a pleckstrin homology domain specific for phosphatidylinositol 4, 5-bisphosphate (PH-GFP) during the cell cycle. Phosphatidylinositol 4, 5-bisphosphate levels are localized in a reciprocal manner, as shown in Janetopoulos et al. Dev Cell 2005. This supports that ARP2/3-mediated actin polymerization takes place at the poles, and formin-mediated assembly in the furrow, where there is actomyosin contraction.

During cytokinesis, plasma membrane PI(4,5)P2 levels rise uniformly as cells round up at metaphase and contribute to the rounding up of the cell. These intermediate levels help reset polarity. PI(4,5)P2 levels subsequently rise in the furrow and are lowered at the poles triggering differential actin assembly, largely through localizing factors specific to the activity of the Rho GTPases, with Arp2/3-mediated filaments forming at the poles. Stimulating metaphase PTEN null cells with chemoattractant gives a transient phosphatidylinositol 3,4,5-trisphosphate and F-actin response. Interestingly, lack of PTEN leads to elevation of phosphatidylinositol 3,4,5-trisphosphate levels, but they are still regulated in response to chemoattractants at metaphase, suggesting that the threshold for Ras activity is different at this stage. These findings have important implications for cancer. Therapeutic strategies should consider targeting a host of enzymes that regulate PI(4,5)P2 levels, as cancers with low plasma mambrane PI(4,5)P2 are likely to be highly metastatic.

PI 4′ and 5′ kinases help terminate uniform chemoattractant-induced responses and contribute to the “backness” of migrating cells. The enzymatic activity that regulates increased PM PI(4,5)P2 levels is likely a major inhibition component in the Local Excitation/Global Inhibition model that regulates chemotaxis. The responses seen during a global stimulation and the spatial distribution of signaling molecules when cells are in a chemical gradient may be explained largely by changes in PM PI(4,5)P2 levels, which influence actin assembly and cell morphology.

### Example 3

### Cells Expressing iRAP System Show PI(4,5)P2 Depletion

FIG. 8 shows the complete remodeling of the cytoskeleton when plasma membrane PI(4,5)P2 levels are depleted using a rapamycin inducible system in mda-mb-231 cancer cell lines. Addition of rapamycin recruits a 5 phosphatase to the plasma membrane. A variety of biosensors were then used to show that stress fibers dissolve, and protrusions and filopodia are extended in many areas of a cell with PI(4,5)P2 depletion. It is proposed that the dropping of PI(4,5)P2 levels leads to local severing (via cofilin and gelsolin) of stress fibers and that this plays a role in the release of AldolaseA, and likely other glycolytic enzymes that are thought to bind F actin. This creates NADH, ATP, and GTP locally and helps drive branching actin networks used for protrusion, and likely activates many other pathways that rely on these nucleotides. Lowering PI(4,5)P2 is likely the driving mechanism underlying the Warburg effect. Cancer cells often have mutations or deletions that activate pathways that use PI(4,5)P2 as a substrate, leading to constant actin remodeling. PI4 and 5 Kinases are turned on with some delay, and raise the PI(4,5)P2 levels following ligand stimulation, helping terminate uniform responses and promoting “backness” and sequestering Aldolase. The Excitation and Inhibition in the Local Excitation, Global Inhibition (LEGI) model we have proposed regulates chemotaxis is largely controlled by the enzymes regulating PI(4,5)P2 levels. This also now has an affect on the local metabolism since glycolysis appears to be activated. The local change in charge driven by changes in PI(4,5)P2 also has a dramatic impact on the redistribution of many other proteins, including the many GEFs that have PH domains with differing phosphoinositide affinities and activate the GTPases at the front and rear of a cell. This is also true in the furrow and poles of a dividing cell.

The inventions described and claimed herein have many attributes and embodiments including, but not limited to, those set forth or described or referenced in this Detailed Description. It is not intended to be all-inclusive and the inventions described and claimed herein are not limited to or by the features or embodiments identified in this Detailed Description, which is included for purposes of illustration only and not restriction.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1-30. (canceled)

31. A method of treating a cancer in a subject comprising administration of an agent that alters the level of phosphatidylinositol-4,5-bisphosphate in a cancer cell of the subject.

32. A method of diagnosing cancer in a subject comprising:
(a) contacting a biological sample with a binding agent that that specifically binds to phosphatidylinositol-4,5-bisphosphate for sufficient time to form a first complex;
(b) removing constituents of the sample;
(c) contacting the first complex with a second binding agent to form a second complex;
(d) determining the level of phosphatidylinositol-4,5-bisphosphate in the sample by detecting the second complex;
wherein lower level of phosphatidylinositol-4,5-bisphosphate in the sample, compared to a control is correlated with cancer.

33. A method for determining the rate of turnover of PI(4,5)P2 levels in a sample, the method comprising:
(a) contacting a sample comprising at least one cancer cell with labelled PI(4,5)P2 at an initial concentration;
(b) incubating the sample under cell growth conditions for an amount of time sufficient to cause turnover of the PI(4,5)P2;
(c) performing an assay to detect the level of one or more of the PI(4,5)P2, phosphatidylinositol-3,4,5-trisphosphate and inositol 1,4,5-trisphosphate;
(d) comparing the level of PI(4,5)P2, phosphatidylinositol-3,4,5-trisphosphate and inositol 1,4,5-trisphosphate, carrying the label, with the initial concentration; and,
(e) determining the rate of turnover of PI(4,5)P2.
